(19) United States
(12) Patent Application Publication
(10) Pub. No.: US 2023/0213510 A1
(43) Pub. Date: Jul. 6, 2023

(54) METHODS RELATING TO IMPROVING ACCURACY OF CAPTURE OBJECT-BASED ASSAYS

(71) Applicant: Quanterix Corporation, Billerica, MA (US)

(72) Inventors: David C. Duffy, Arlington, MA (US); David M. Rissin, Lexington, MA (US); Matthew Fishburn, Somerville, MA (US); Andrew Rivnak, Somerville, MA (US); Purvish Patel, Arlington, MA (US)

(73) Assignee: Quanterix Corporation, Billerica, MA (US)

(21) Appl. No.: 18/077,878

(22) Filed: Dec. 8, 2022

Related U.S. Application Data
(63) Continuation of application No. 15/543,401, filed on Jul. 13, 2017, now abandoned.
(60) Provisional application No. 62/102,818, filed on Jan. 13, 2015.

(51) Int. Cl. G01N 33/543

(52) U.S. Cl. CPC G01N 33/54393; G01N 33/54306

(57) ABSTRACT

Described herein are methods for improving the accuracy of capture object-based assays. In some embodiments, a measure of the number or a measure of the concentration of an analyte molecule or particle in a fluid sample is determined using the capture object-based assay.

### FIELD OF THE INVENTION

Described herein are methods for improving the accuracy of capture object-based assays.

In some embodiments, a measure of the number and/or a measure of the concentration of an analyte molecule or particle in a fluid sample is determined using the capture object-based assay.

### BACKGROUND OF THE INVENTION

The ability to precisely measure target analyte molecules (e.g., proteins) is important in several fields, including clinical diagnostics, testing of blood banks, and the analysis of biochemical pathways. Assays exist for the simultaneous detection of single molecules of target analyte molecules, which may utilize beads or other capture objects (e.g., digital ELISA, see Rissin et al., Nat. Biotechnol. 2010, 28, 595-599, herein incorporated by reference). Certain digital ELISA assays involve capturing proteins on microscopic beads (or other capture objects), labeling the target analytes with an enzyme, isolating the beads in arrays of small wells, and detecting bead-associated enzymatic activity using fluorescence imaging. Spatial localization and/or separation of individual beads, for example in arrays, enables the simultaneous determination of the single molecule signal associated the beads, enabling a measure of the number and/or concentration of the target analyte to be determined at very low values. Various other capture object-based assay have also been developed to determine a measure of the number and/or concentration of analyte molecules in a fluid sample, wherein the analyte molecules are captured on beads or other capture objects. However, there is a continued need for methods and techniques to improve the accuracy and sensitivity of these capture object-based assays.

### SUMMARY OF THE INVENTION

Described herein are methods for improving the accuracy of capture object-based assays. In some embodiments, a measure of the number and/or the concentration of an analyte molecule or particle in a fluid sample is determined using the capture object-based assay. The subject matter of the present invention involves, in some cases, interrelated products, alternative solutions to a particular problem, and/or a plurality of different uses of one or more systems and/or articles.

In some embodiments, a method for determining a measure of the concentration of analyte molecules or particles in a fluid sample is provided comprising: exposing a plurality of capture objects to a solution containing or suspected of containing a first type of analyte molecules or particles, wherein the capture objects comprise a first type of capture object and one or more types of non-targeting capture objects; wherein each of the first type of capture object includes a binding surface having specific binding affinity for the first type of analyte molecule or particle; wherein each of the one or more types of non-targeting capture objects do not include any binding surfaces having specific binding affinity for any type of analyte molecules or particles contained in or suspected to be contained in the solution; wherein the ratio of the number of first type of capture objects to the total number of capture objects is between 1:1.2 and 1:100; and wherein at least some of the first type of capture objects associate with at least one analyte molecule or particle and at least some of the first type of capture objects do not associate with any analyte molecules or particles; spatially separating at least a portion of the plurality of capture objects subjected to the exposing step into a plurality of separate locations; addressing at least some of the plurality of locations and determining the number of locations containing a first type of capture object; further determining the number of said locations containing a first type of capture object and a first type of analyte molecule or particle; and determining a measure of the concentration of the first type of analyte molecules or particles in the fluid sample based at least in part on the ratio of the number of locations containing a first type of capture object and a first type of analyte molecule and particle, to the number of locations containing a first type of capture object.

In some embodiments, a method for determining a measure of the concentration of a first type of analyte molecules or particles in a fluid sample is provided comprising exposing a plurality of capture objects to a solution containing or suspected of containing the first type of analyte molecules or particles, wherein the capture objects comprise a first type of capture object and one or more types of non-targeting capture objects; wherein each of the first type of capture object includes a binding surface having specific binding affinity for the first type of analyte molecule or particle; wherein each of the one or more types of non-targeting capture objects do not include any binding surfaces having specific binding affinity for the first type of analyte molecules or particles; wherein the ratio of the first type of capture objects to the total number of capture objects is between 1:1.2 and 1:100; and wherein at least some of the first type of capture objects associate with at least one analyte molecule or particle and at least some of the first type of capture objects do not associate with any analyte molecules or particles; spatially separating at least a portion of the plurality of capture objects subjected to the exposing step into a plurality of separate locations; addressing at least some of the plurality of locations and determining the number of locations containing a first type of capture object; further determining the number of said locations containing a first type of capture object and a first type of analyte molecule or particle; and determining a measure of the concentration of only the first type of analyte molecules or particles in the fluid sample based at least in part on the ratio of the number of locations containing a first type of capture object and a first type of analyte molecule and particle, to the number of locations containing a first type of capture object.

In some embodiments, a method for determining a measure of the concentration of only a first type and a second type of analyte molecules or particles in a fluid sample is provided comprising exposing a plurality of capture objects to a solution containing or suspected of containing first types of analyte molecules or particles and a second type of analyte molecules or particles, wherein the capture objects comprise a first type of capture object, a second type of capture object, and one or more types of non-targeting capture objects; wherein each of the first type of capture object includes a binding surface having affinity for the first type of analyte molecule or particle; wherein each of the second type of capture object includes a binding surface having affinity for the second type of analyte molecule or particle; and wherein each of the one or more types of non-targeting capture objects do not include any binding surfaces having affinity for the first type of analyte molecules or particles or the second type of analyte molecules or particles; wherein the ratio of the first type of capture objects to the total number of capture objects and the ratio of the second type of capture objects to the total number of capture objects are the same or different and are between 1:1.2 and 1:100; wherein at least some of the first type of capture objects associate with at least one analyte molecule or particle and at least some of the first type of capture objects do not associate with any analyte molecule or particle; and wherein at least some of the second type of capture objects associate with at least some of the second type of analyte molecule or particle at least some of the second type of capture objects do not associate with any analyte molecule or particle; spatially separating at least a portion of the capture objects subjected to the exposing steps into a plurality of separate locations; addressing at least some of the plurality of locations and determining the number of locations containing a first type capture object or a second type of capture object; further determining the number of said locations containing a first type of analyte molecule or particle or a second type of analyte molecule or particle; and determining a measure of the concentration of only the first type of analyte molecules or particles and the second type of analyte molecules or particles in the fluid sample based at least in part on the ratio of the number of locations containing a first type of capture object and a first type of analyte molecule and particle, to the number of locations containing a first type of capture object, or based at least in part on the ratio of the number of locations containing a second type of capture object and a second type of analyte molecule and particle, to the number of locations containing a second type of capture object, respectively.

In some embodiments, a method for binding analyte molecules or particles in a fluid sample to capture objects and spatially separating the capture objects is provided comprising exposing a plurality of capture objects to a solution comprising or derived from the fluid sample, wherein the capture objects comprise at least one type of targeting capture objects and at least one type of non-targeting capture objects; wherein each of the at least one type of targeting capture objects includes a binding surface having specific binding affinity for at least one type of target analyte molecule or particle contained in or suspected to be contained in the solution, wherein each of the one or more types of non-targeting capture objects do not include any binding surfaces having specific binding affinity for any of the at least one type of target analyte molecule or particle contained in or suspected to be contained in the solution, wherein the ratio of the number of targeting capture objects to the total number of targeting and non-targeting capture objects is between 1:1.2 and 1:100; spatially separating at least a portion of the plurality of capture objects subjected to the exposing step.

In some embodiments, a method for determining a measure of the concentration of a first type of analyte molecules or particles in a fluid sample is provided comprising exposing a plurality of capture objects to a solution containing or suspected of containing a first type of analyte molecules or particles, wherein the capture objects comprise a first type of capture object and a second type of capture object; exposing the plurality of capture objects to a second type of analyte molecules or particles, wherein: each of the first type of capture object includes a binding surface having specific binding affinity for the first type of analyte molecule or particle; each of the second type of capture objects do not include any binding surfaces having specific binding affinity for the first type of analyte molecules or particles contained in or suspected to be contained in the solution and include at least one binding surface having some affinity for the second type of analyte molecule or particle; at least some of the first type of capture objects associate with at least one analyte molecule or particle and at least some of the first type of capture objects do not associate with any analyte molecules or particles, a statistically significant fraction of the second type of capture objects associate with either zero or one of the second type of analyte molecules or particles, and the ratio of the number of first type of capture objects to the total number of capture objects is between 1:1.2 and 1:100; spatially separating at least a portion of the plurality of capture objects subjected to the exposing step into a plurality of separate locations; addressing at least some of the plurality of locations and determining the number of locations containing a first type of capture object; further determining the number of the locations determined to contain a first type of capture object that also contain a first type of analyte molecule or particle; addressing at least some of the plurality of locations and determining which locations contain a second type of capture object and a binding ligand; further determining the average intensity of said locations containing a second type of capture object and a second type of analyte molecule or particle; and determining a measure of the concentration of the first type of analyte molecules or particles in the fluid sample based at least in part on the average intensity of said locations containing a second type of capture object and a second type of analyte molecule or particle.

### BRIEF DESCRIPTION OF THE DRAWINGS

FIGS. 1, 2, and 3 illustrate non-limiting examples of methods comprising a plurality of targeting capture objects and a plurality of non-targeting capture objects, according to some embodiments; and

FIG. 4 shows plots of the number of beads loaded into a plurality of reaction vessels for a number of samples utilizing A) only targeting beads and b) targeting beads and non-targeting beads.

Other aspects, embodiments, and features of the invention will become apparent from the following detailed description when considered in conjunction with the accompanying drawings. The accompanying figures are schematic and are not intended to be drawn to scale. For purposes of clarity, not every component is labeled in every figure, nor is every component of each embodiment of the invention shown where illustration is not necessary to allow those of ordinary skill in the art to understand the invention. All patent applications and patents mentioned in the text are incorporated by reference in their entirety. In case of conflict between the description contained in the present specification and a document incorporated by reference, the present specification, including definitions, will control.

### DETAILED DESCRIPTION

Described herein are methods for improving the accuracy of capture object-based assays, wherein a measure of the number and/or a measure of the concentration of analyte molecules or particles in a fluid sample is determined using the capture object-based assays. The subject matter of the present invention involves, in some cases, interrelated products, alternative solutions to a particular problem, and/or a plurality of different uses of one or more systems and/or articles. It should be understood, that while much of the discussion below is directed to assays utilizing beads as the capture objects, this is by way of example only, and other types of capture objects may be employed. In addition, while much of the discussion below is directed to assays utilizing target analyte molecules as the analyte of interest, this is by way of example only, and other types of target analytes may be employed, for example, target analyte particles.

In some embodiments, methods for determining a measure of the number and/or a measure of the concentration of analyte molecules in a fluid sample are provided, wherein the method comprises the steps of associating the analyte molecules or particles with a plurality of capture objects (e.g., beads) and spatially separating at least a portion of the plurality of capture objects. Following spatial separation, a portion of the spatially separated capture objects are addressed to determine a measure of the number of the analyte molecules associated with the portion of the capture objects addressed. In some embodiments, a portion of the spatially separated capture objects are addressed to determine a measure of the number and/or a measure of the concentration of the analyte molecules in the fluid sample.

Without wishing to be bound by theory, the advantages may be caused by a balancing of factors. For examples, in some embodiments, it was determined that the number of capture objects initially added to a fluid sample for target analyte capture could be both advantageous and disadvantageous with respect to changes in sensitivity. For example, when using fewer beads, the ratio of target analyte to capture objects increases, the signal (average target analyte per capture object) increases, and therefore, the assay sensitivity generally increases. However, using fewer capture objects also reduces the number of capture objects that may be spatially separated (e.g., into a plurality of locations), and, if that number drops to a level where Poisson noise becomes significant, then the quantitation of capture objects can become noisy and sensitivity may be decreased. The use of both targeting and non-targeting capture object counters this dilemma by allowing the minimum number of capture objects to be used to increase average target analyte per capture object while keeping the capture loading number as high and/or consistent as possible.

In many embodiments, the methods described herein utilize a combination of targeting capture objects and non-targeting capture objects. For example, in some embodiments, the plurality of capture objects comprise a plurality of types of capture objects, wherein at least one type of capture object is a targeting capture object which includes a binding surface having specific binding affinity for at least one type of target analyte molecule or particle contained in or suspected to be contained in the fluid sample and at least one type of capture object is a non-targeting capture object. In some embodiments, the methods described herein comprising the use of both targeting and non-targeting capture objects provide advantages over previously described methods utilizing only targeting capture objects. For example, in some embodiments, the methods described herein comprising the use of both targeting and non-targeting capture objects may result in a lower coefficient of variation of the number of targeting capture objects determined to be associated with a target analyte as compared to a substantially similar methods utilizing only targeting capture objects.

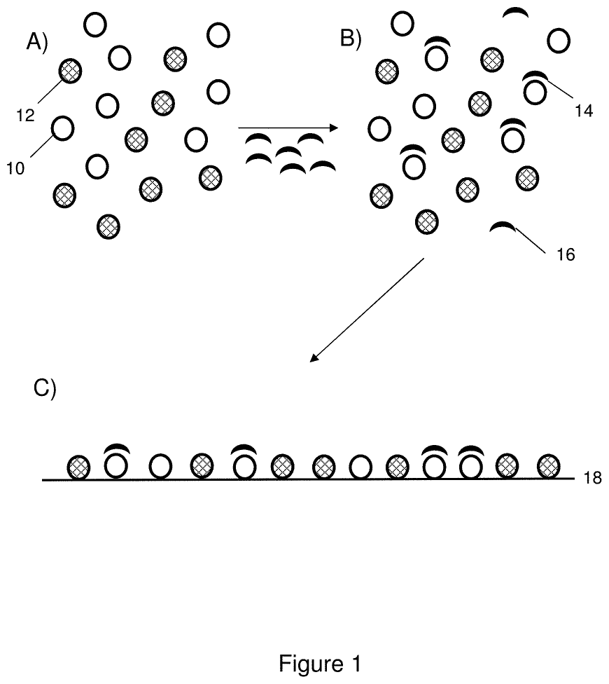

A non-limiting assay method is depicted in FIG. 1. In Step A, a plurality of types of capture objects are provided comprising first type of capture object 10 having specific binding affinity for a first type of target analyte molecule and second type of capture object 12 being non-targeting capture objects. The plurality of capture objects are exposed to a fluid sample comprising the first type of analyte molecule. At least some of the first type of analyte molecules (e.g., 14) associate with a first type of capture object, as shown in Step B. In some embodiments, at least some of the first type of analyte molecules (e.g., 16) do not associate with any capture objects. At least a portion of the capture objects from Step B are then spatially separated, for example, into a plurality of locations. For example, as shown in Step C, a portion of the capture objects are spatially separated by association with surface 18. Other methods for spatially separating the capture objects are described herein. Following spatial separation, a measure of the number of and/or a measure of the concentration of the first type of analyte molecules in the fluid sample may be determined. For example, a portion of the capture objects spatially separated may be addressed to determine the number of the first type of analyte molecule associated with a first type of capture object. In some embodiments, the measure of the concentration of the first type of analyte molecule may be determined at least in part based on the number of the first type of capture object determined to be associated with a first type of analyte molecule and/or the ratio of the number of the first type of capture object associated with a first type of analyte molecule, to the total number of first type of capture objects.

The assay methods described herein may employ a variety of different components, steps, and be used for a variety of different purposes as described herein. Details of certain exemplary methods and exemplary components utilized by certain of the methods will now be discussed in detail. It should be understood, that none, a portion of, or all of the following steps may be performed at least once during certain exemplary method formats described herein and/or none, a portion, or all of the components may be utilized at least once during certain exemplary method formats described herein. Non-limiting examples of additional steps not described which may be performed include, but are not limited to, washing steps, exposure to additional reagents, and/or various sample/data analysis steps. In some cases, the methods may include the use of at least one binding ligand, as described herein. In some cases, the measure of the concentration of analyte molecules in a fluid sample is based at least in part on comparison of a measured parameter to a calibration curve. In some instances, the calibration curve is formed at least in part by determination at least one calibration factor, as described herein. In some cases, a method may further comprise at least one background signal determination (e.g., and further comprise subtracting the background signal from other determinations).

As used herein, the term targeting capture object refers to a capture object which includes a binding surface having specific binding affinity for at least one type of target analyte molecule or particle contained in or suspected of being contained in a solution to be tested. That is, a targeting capture object is a capture object which includes one or more surfaces which are selected so as to specifically bind a target analyte. The term specific binding affinity, as used herein, refers to the ability of one substance (e.g., capture object) to specifically bind to a first substance (e.g., target analyte) with a high association constant (i.e., >10⁶ M⁻¹) and with high specificity over other substances (i.e., <10⁶ M⁻¹ association constant other target analytes). In some embodiments, a target analyte may become immobilized with respect to the targeting capture object. As used herein, the term immobilized means captured, attached, bound, or affixed so as to reduce dissociation or loss of the target analyte molecule, but does not require absolute immobility with respect to the capture object.

Those of ordinary skill in the art will be aware of suitable targeting capture objects to be used in the methods described herein. For example, in some cases, the targeting capture object comprises at least one surface comprising a plurality of targeting entities. As used therein, the term “targeting entity” is any molecule or other chemical/biological entity that can be used to impart specific binding affinity for a target molecule or particle (e.g., an analyte molecule), such that the target analyte molecule becomes immobilized with respect to the targeting entity. The immobilization, as described herein, may be caused by the association of an analyte molecule with the targeting entity.

The selection of the targeting entity will depend on the composition of what is being targeted (e.g., the target analyte molecule or particle). Targeting entities for a wide variety of target analyte molecules are known or can be readily found or developed using known techniques. For example, when the target analyte molecule is a protein, the targeting entity may comprise proteins, particularly antibodies or fragments thereof (e.g., antigen-binding fragments (Fabs), Fab′ fragments, pepsin fragments, F(ab′)₂ fragments, full-length polyclonal or monoclonal antibodies, antibody-like fragments, etc.), other native or recombinant proteins, such as receptor proteins, Protein A, Protein G, Protein C, avidin, streptavidin, etc., or small molecules, such as, for example biotin. In some cases, targeting entities for proteins comprise peptides. For example, when the target analyte molecule is an enzyme, suitable targeting entities may include enzyme substrates and/or enzyme inhibitors. In some cases, when the target analyte molecule is a phosphorylated species, the targeting entity may comprise a phosphate-binding agent. In addition, when the target analyte molecule is a single-stranded nucleic acid, the targeting entity may be a complementary nucleic acid. Similarly, the target analyte molecule may be a nucleic acid binding protein and the targeting entity may be a single-stranded or double-stranded nucleic acid; alternatively, the targeting entity may be a nucleic acid-binding protein when the target molecule is a single or double stranded nucleic acid. Also, for example, when the target analyte molecule is a carbohydrate, potentially suitable targeting entity include, for example, antibodies, lectins, and selectins. As will be appreciated by those of ordinary skill in the art, any molecule that can specifically associate with a target analyte molecule of interest may potentially be used as a targeting entity. For certain embodiments, suitable target analyte molecule/targeting entity pairs can include, but are not limited to, antibodies/antigens, antigens/antibodies, receptors/ligands, proteins/nucleic acid, nucleic acids/proteins, nucleic acids/nucleic acids, enzymes/substrates and/or inhibitors, carbohydrates (including glycoproteins and glycolipids)/lectins and/or selectins, proteins/proteins, proteins/small molecules; small molecules/small molecules, etc.

In some embodiments, a targeting capture object includes a binding surface having plurality of targeting entities. The portion of the object which comprises a binding surface may be selected or configured based upon the physical shape/characteristics and properties of the objects (e.g., size, shape), and the format of the assay. In some embodiments, substantially all of the outer surfaces of the object comprise a plurality of targeting entities. According to one embodiment, each binding surface of an object comprises a plurality of targeting entities. The plurality of targeting entities, in some cases, may be distributed randomly on the binding surface like a “lawn.” Alternatively, the targeting entities may be spatially separated into distinct region(s) and distributed in any desired fashion or pattern.

As used herein, the term “non-targeting capture object” refers to a capture object which does not include any binding surfaces having specific binding affinity for at least one, some or all of the target analyte molecules or particles contained in or suspected to be contained in a test solution whose amount/concentration is to be determined with an assay that employs such capture objects. In some embodiments, wherein a first type of analyte molecule is to be detected, the non-targeting capture objects do not include any binding surfaces having specific binding affinity for the first type of target analyte. That is, the non-targeting capture objects do not include any surfaces to which a target analyte would specifically bind, however, such capture objects may include binding surfaces having specific binding affinity for other types of analytes which may be contained or suspected to be contained in the fluid sample. For example, in some embodiments, wherein target molecule B is to be detected, a method may employ non-targeting capture objects A comprising at least one surface comprising targeting entities A which may be utilized to immobilize analyte A. In some embodiments, at least one type of non-targeting capture object does not have specific affinity for a first type of target analyte molecule or particle whose concentration or amount is to be determined with the assay but may include at least one binding surface having specific affinity for a second type of target analyte molecule or particle. In some embodiments, the concentration of the second type of target analyte is not determined. In some embodiments, the second type of target analyte is detected for other reasons (e.g., to calibrate the system, etc.), as described in more detail herein. In other embodiments, wherein a first type of analyte molecule is to be detected, the non-targeting capture objects do not include any binding surfaces having specific binding affinity for any type of analyte molecule or particle contained or suspected of being contained in the fluid sample. For example, in some embodiments, wherein analyte molecules A, B, C, and D are contained or suspected to be contained in the fluid sample, the non-targeting capture objects do not include any binding surfaces having specific binding affinity for analyte molecules A, B, C, or D.

The ratio of the number of each type of targeting capture objects to the total number of targeting and non-targeting capture objects may be any suitable value. In some cases, the ratio may be between 1:1.2 and 1:100, or between 1:2 and 1:100, or between 1:5 and 1:100, or between 1:5 and 1:75, or between 1:5 and 1:50, or between 1:5 and 1:25, or between 1:10 and 1:100, or between 1:10 and 1:75, or between 1:10 and 1:50, or between 1:20 and 1:100, or between 1:25 and 1:100. In embodiments where more than one type of targeting capture object are utilized, the above ratios may apply to each of the types of targeting capture objects. For example, wherein a first type and a second type of targeting capture objects are utilized, the ratio of the first type of targeting capture object to the total number of capture objects (e.g., the total number of the first type of capture object, the second type of capture object, and all non-targeting capture objects) and the ratio of the second type of targeting to the total number of capture objects may be between 1:1.2 and 1:100, or between 1:2 and 1:100, or between 1:5 and 1:100, or between 1:5 and 1:75, or between 1:5 and 1:50, or between 1:5 and 1:25, or between 1:10 and 1:100, or between 1:10 and 1:75, or between 1:10 and 1:50, or between 1:20 and 1:100, or between 1:25 and 1:100.

Additional non-limiting methods comprising the use of both targeting and non-targeting capture objects will now be described in detail.

In a first non-limiting embodiment, methods for determining a first type of target analyte are provided, wherein the method comprising non-targeting capture objects that do not have any affinity for other molecules in the solution are provided. In some embodiments, a method for determining a measure of the number and/or the concentration of analyte molecules or particles in a fluid sample (or a solution derived from the fluid sample) comprises exposing a plurality of capture objects to a solution containing or suspected of containing a first type of analyte molecules or particles, wherein the plurality of capture objects comprise a first type of capture object and one or more types of non-targeting capture objects. In this non-limiting method, each of the first type of capture object includes a binding surface having specific binding affinity for the first type of analyte molecule or particle and each of the one or more types of non-targeting capture objects do not include any binding surfaces having specific binding affinity for any type of analyte molecules or particles contained in or suspected to be contained in the solution. That is, at least a portion of the capture object contain a binding surface having specific binding affinity to the first type of analyte molecule or particle (e.g., targeting capture objects for the first type of analyte molecule or particle) and at least a portion of the capture objects do not contain any binding surfaces having specific binding affinity for any molecules or particles contained in the fluid sample (e.g., the first type of analyte molecule and any other molecules known or suspected to be contained in the fluid sample). For example, in embodiments where fluid sample contains or is suspected to contain two types of analyte molecules (e.g., analyte molecules A and B), the non-targeting capture objects would not include any binding surfaces having specific binding affinity for either type of analyte molecule (e.g., analyte molecules A and B). The ratio of the first type of capture object to the total number of capture objects (e.g., first type of capture object and any non-targeting capture objects) may be between 1:1.2 to 1:100, or between 1.5 to 1:100, or any other ratio described herein. Following and/or during the exposing step, at least some of the first type of capture objects associate with at least one analyte molecule or particle while in certain cases at least a some of the first type of capture objects do not associate with any analyte molecules or particles. At least a portion of the capture objects which were subjected to the exposing step may then be spatially separated, for example, into a plurality of separate locations, and analyzed to determine the total number of the first type of capture objects associated with a first type of analyte molecule or particle. In some embodiments, the number of locations containing a first type of capture object (e.g., whether associated or not associated with a first type of capture object) and the number of locations containing a first type of capture object associated with a first type of analyte molecule are both determined. The number of locations which contain a non-targeting capture object may or may not be determined. In embodiments where a measure of the concentration of the first type of target analyte in a fluid sample is to be determined, the measure of the concentration of the first type of target analyte may be determined based at least in part on the ratio of the number of locations containing a first type of capture object associated with a first type of analyte molecule to the total number of locations containing a first type of capture object, as described herein.

This first non-limiting embodiment may be described with reference to FIG. 1. In Step A, a plurality of types of capture objects are provided comprising first type of capture object 10 having specific binding affinity of a first type of target analyte molecule and second type of capture object 12 being non-targeting capture objects. Each of first type of capture object 10 includes a binding surface having specific binding affinity for the first type of analyte molecule or particle and each of the second type of capture object 12 does not include any binding surfaces having specific binding affinity for any type of analyte molecules or particles contained in or suspected to be contained in the solution (e.g., the first type of analyte molecule and any other molecules known or suspected to be contained in the fluid sample). The capture objects are exposed to a fluid sample comprising the first type of analyte molecule and at least some of the analyte molecules (e.g., 14) associate with a first type of capture object, as shown in Step B. In some embodiments, at least some of the analyte molecules (e.g., 16) do not associate with any capture objects. At least a portion of the capture objects from Step B are then spatially separated, for example, into a plurality of locations. For example, as shown in Step C, a portion of the capture objects are spatially separated by association with surface 18. Other methods for spatially separating the capture objects are described herein. Following spatial separation, a measure of the number and/or a measure of the concentration of the analyte molecules in the fluid sample may be determined. In some embodiments, at least some of the plurality of capture objects subjected to the spatially separating are addressed to determine the number of locations containing a first type of capture object (e.g., whether associated or not associated with a first type of analyte) and/or to determine the number of locations containing a first type of capture object associated with a first type of analyte molecule. The number of locations containing a non-targeting capture object may or might not be determined. In embodiments where a measure of the concentration of the first type of target analyte in a fluid sample is to be determined, the measure of the concentration of the first type of target analyte may be determined at least in part on the ratio of the number of locations containing a first type of capture object associated with a first type of analyte molecule to the total number of locations containing a first type of capture object.

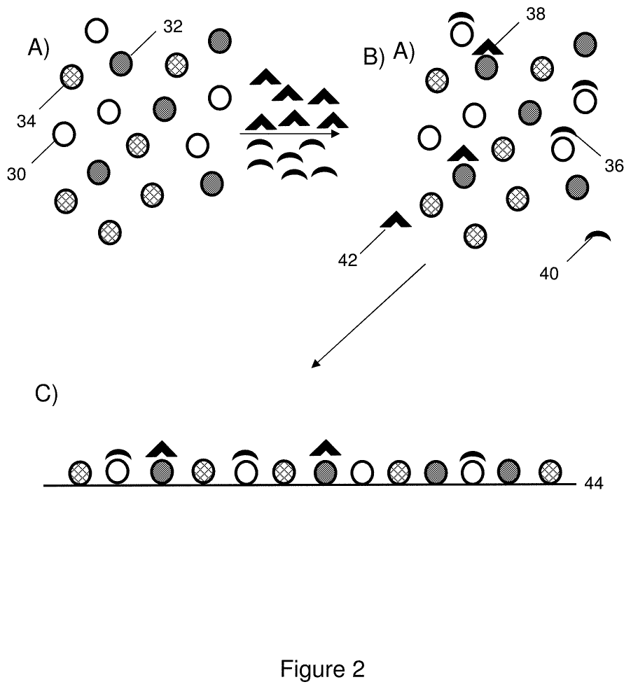

It should be understood, that for the first embodiment described above, more than one type of analyte molecule may be determined. For example, a first type and a second type of analyte molecule may be determined. A non-limiting example of such a method is depicted in FIG. 2. In Step A, a plurality of types of capture objects are provided comprising a first type of capture object 30 having specific binding affinity for a first type of target analyte molecule, a second type of capture object 32 having specific binding affinity for a second type of target analyte molecule, and a third type of capture object 34 being non-targeting capture objects. Each of the first type of capture object 30 includes a binding surface having specific binding affinity for the first type of analyte molecule or particle, each of the second type of capture object 32 includes a binding surface having specific binding affinity for the second type of analyte molecule or particle, and each of the third type of capture object 34 do not include any binding surfaces having specific binding affinity for the first type or the second type of analyte molecules or particles contained in or suspected to be contained in the solution. The capture objects are exposed to a fluid sample comprising the first type of analyte molecule and the second type of analyte molecule. As shown in Step B, at least some of the first type of analyte molecules (e.g., 36) associate with a first type of capture object and at least some of the second type of analyte molecules (e.g., 38) associate with a second type of capture object. In some embodiments, at least some of the first type and the second type analyte molecules (e.g., 40 and 42, respectively) do not associate with any capture objects. At least a portion of the capture objects from Step B are then spatially separated, for example, in to a plurality of locations. For example, as shown in Step C, a portion of the capture objects are spatially separated by association with surface 44.

Other methods for spatially separating the capture objects are described herein. Following spatial separation, a measure of the number of and/or a measure of the concentration of the first type and/or the second type analyte molecules in the fluid sample may be determined. In some embodiments, at least some of the plurality of capture objects subjected to the spatial separation step are addressed to determine the number of location containing a first type of capture object (e.g., whether associated or not associated with a first type of target analyte), the number of location containing a second type of capture object (e.g., whether associated or not associated with a second type of target analyte), the number of locations containing a first type of capture object associated with a first type of analyte molecule, and the number of locations containing a second type of capture object associated with a second type of analyte molecule. In embodiments where a measure of the concentration of the first type of target analyte in the fluid sample is to be determined, the measure of the concentration of the first type of target analyte may be determined at least in part on the ratio of the number of locations containing a first type of capture object associated with a first type of analyte molecule to the total number of locations containing a first type of capture object. In embodiments where a measure of the concentration of the second type of target analyte in the fluid sample is to be determined, the measure of the concentration of the second type of target analyte may be determined at least in part on the ratio of the number of locations containing a second type of capture object associated with a second type of analyte molecule to the total number of locations containing a second type of capture object. Those of ordinary skill in the art will be able to apply these teachings to methods for determining more than two types of target analytes, for example, three types, or four types, or more.

In a second non-limiting embodiment, methods for determining only a first type a target analyte are provided, wherein the method comprises non-target capture objects which may or might not have affinity for other molecules known or suspected to be present in the solution. For example, in some embodiments, a method for determining a measure of the concentration of only a first type of analyte molecules or particles in a fluid sample are provided. In some cases, the method comprises exposing a plurality of capture objects to a solution containing or suspected of containing the first type of analyte molecules or particles. In this embodiment, the capture objects comprise a first type of capture object and one or more types of non-targeting capture objects; wherein each of the first type of capture object includes a binding surface having specific binding affinity for the first type of analyte molecule or particle and each of the one or more types of non-targeting capture objects do not include any binding surfaces having specific binding affinity for the first type of analyte molecules or particles. Accordingly, in this embodiment, the one or more types of non-targeting capture objects may optionally have a binding surface which has specific binding affinity for another type of molecule or particles contained or suspected to be contained in the fluid sample, but do not have specific binding affinity for the first type of analyte molecule being assayed. The ratio of the first type of capture objects to the total number of capture objects may be between 1:1.2 and 1:100, or between 1:5 and 1:100 or any ratio described herein. Following and/or during the exposing step, at least some of the first type of capture objects associate with at least one first type of analyte molecule or particle and at least some of the first type of capture objects may not associate with any of the first type of analyte molecules or particles. Similar steps as described in the previous exemplary embodiment may then be carried out using the capture objects subjected to the exposing step. For example, in some embodiments, at least a portion of the plurality of capture objects subjected to the exposing step are spatially separated, for example, into a plurality of separate locations. At least some of the plurality of locations may be addressed to determine the number of locations containing a first type of capture object and/or the number of locations containing a first type of analyte molecule associated with a first type of capture object. Furthermore, a measure of the concentration of only the first type of analyte molecules or particles in the fluid sample may be determined based at least in part on the ratio of the number of locations containing a first type of capture object associated with a first type of analyte molecule and particle, to the number of locations containing a first type of capture object.

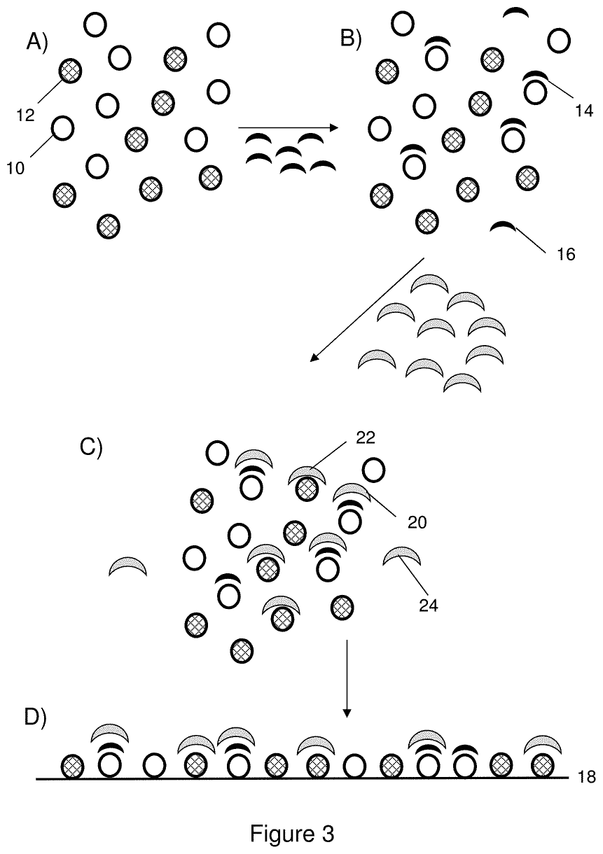

As a specific non-limiting example of the second non-limiting embodiment, methods for determining a first type a target analyte may comprise use of at least one type of non-target capture object which has affinity for a second type of target analyte. In some embodiments, the concentration of the second type of target analyte is not determined. In some embodiments, the second type of target analyte may be detected for other reasons. In some embodiments, the second type of target analyte is a binding ligand. The binding ligand may be utilized to determine the presence or absence of a first type of target analyte. In other embodiments, the second type of target analyte may be a non-target molecule which is known or suspected to be present in the solution, but for which the unknown concentration is not required to be determined. For example, the non-target molecules may be used to calibrate or otherwise provide information to be used in the assay, but the concentration of the non-target molecules need not be determined. In some cases, the method comprises exposing a plurality of capture objects to a solution containing or suspected of containing the first type of analyte molecules or particles. In this non-limiting embodiment, the capture objects comprise a first type of capture object and at least one type of non-targeting capture objects; wherein each of the first type of capture object includes a binding surface having specific binding affinity for the first type of analyte molecule or particle and at least one type of non-targeting capture object that includes a binding surface does not have specific binding affinity for the first type of analyte molecules or particles but has specific binding affinity for a non-target molecule. This non-limiting embodiments is depicted in FIG. 3. In Step A, a plurality of types of capture objects are provided comprising first type of capture object 10 having specific binding affinity for a first type of target analyte molecule and second type of capture object 12 being non-targeting capture objects having specific affinity for a second type of target analyte. In this embodiment, the second type of target analyte is a binding ligand used in the detection of the first type of analyte molecule. In addition, the concentration of the second target analyte molecule is not determined in this assay, but rather the second target analyte is detected for other purposes. In this non-limiting example, the second type of target analyte is used to assist in or facilitate the concentration determination of the first type of analyte molecule. The plurality of capture objects are exposed to a fluid sample comprising the first type of analyte molecule. At least some of the first type of analyte molecules (e.g., 14) associate with a first type of capture object, as shown in Step B. In some embodiments, at least some of the first type of analyte molecules (e.g., 16) do not associate with any capture objects. The plurality of capture objects are exposed to a fluid sample comprising the second type of analyte molecule, e.g., a binding ligand having affinity for the first type of analyte molecule. At least some of the first type of analyte molecules associated with a first type of capture object also associate with a binding ligand (e.g., 20), at least some of the second type of capture object associate with the binding ligand (e.g., 22), as shown in Step C. In some embodiments, at least some of the second type of analyte molecules (e.g., 24) do not associate with any capture objects. At least a portion of the capture objects from Step C are then spatially separated, for example, into a plurality of locations. For example, as shown in Step D, a portion of the capture objects are spatially separated by association with surface 18. Other methods for spatially separating the capture objects are described herein. Following spatial separation, a measure of the number of and/or a measure of the concentration of the first type of analyte molecules in the fluid sample may be determined. For example, a portion of the capture objects spatially separated may be addressed to determine the number of the first type of analyte molecule associated with a first type of capture object via detection of the binding ligand. In addition, the number of locations containing a second type of capture object and a binding ligand may also be determined. In some cases, the average intensity of those locations may be determined, and the measure of the concentration of the first type of analyte molecule may be determined at least in part based on the average intensity.

As would be understood by one of ordinary skill in the art, and as described herein in more detail elsewhere, the analyte molecules and/or binding ligands may be directly detectable or indirectly detectable. In some embodiments in which the binding ligand is indirectly detectable, the binding ligand comprises an enzymatic component and/or may be further exposed to a secondary binding ligand comprising an enzymatic component. The binding ligand (or secondary binding ligand) may then be exposed to an precursor labeling agent (e.g., an enzymatic substrated) which is converted to a labeling agent (e.g., a detectable product) upon exposure to the enzymatic component.

Similar to the method described above in the second non-limiting embodiment, described below is a third non-limiting embodiment involving a method for determining a measure of the concentration of only a first type and a second type of analyte molecules or particles in a fluid sample. In this non-limiting embodiment, a plurality of capture objects are exposed to a solution containing or suspected of containing a first type of analyte molecules or particles and a second type of analyte molecules or particles, wherein the capture objects comprise a first type of capture object, a second type of capture object, and one or more types of non-targeting capture objects. Each of the first type of capture object includes a binding surface having affinity for the first type of analyte molecule or particle, each of the second type of capture object includes a binding surface having affinity for the second type of analyte molecule or particle, and each of the one or more types of non-targeting capture objects do not include any binding surfaces having affinity for the first type of analyte molecules or particles or the second type of analyte molecules or particles. Accordingly, in this embodiment, the one or more types of non-targeting capture objects may have a binding surface which optionally has specific binding affinity for another type of analyte molecule or particles contained or suspected to be contained in the fluid sample, but do not have specific binding affinity for the first type or the second type of analyte molecule or particle. For example, at least one of the one or more types of non-targeting capture object may have a binding surface which has specific binding affinity for a non-target molecule (e.g., a molecule for which the concentration of the non-target molecule is not required to be determined for the assay and/or wherein the non-target molecule is used for another purpose (e.g., to calibrate the system/assay, etc.)). The ratio of the first type of capture objects to the total number of capture objects and the ratio of the second type of capture objects to the total number of capture objects are the same or different and may be between 1:1.2 and 1:100, or any ratio described herein. Following and/or during exposure, at least some of the first type of capture objects associate with at least one analyte molecule or particle while at least some of the first type of capture objects may not associate with any analyte molecule or particle, and at least some of the second type of capture objects associate with at least some of the second type of analyte molecule or particle while at least some of the second type of capture objects may not associate with any analyte molecule or particle. Similar steps as described in the above described two exemplary embodiments may then be carried out using the capture objects subjected to the exposing step. For example, at least a portion of the capture objects subjected to the exposing steps may be spatially separated into a plurality of separate locations. At least a portion of the plurality of locations may be addressed to determine the number of locations containing a first type of capture object, the number of locations containing a first type of capture object and a first type of analyte molecule or particle, the number of locations containing a second type of analyte molecule or particle, and/or the number of locations containing a second type of analyte molecule or particle and a second type of capture object. Optionally, a measure of the concentration of the first type of analyte molecules or particles and the second type of analyte molecules or particles in the fluid sample based at least in part on the ratio of the number of locations containing a first type of capture object associated with a first type of analyte molecule and particle, to the number of locations containing a first type of capture object, or based at least in part on the ratio of the number of locations containing a second type of capture object associated with a second type of analyte molecule and particle, to the number of locations containing a second type of capture object, respectively. Those of ordinary skill in the art will be able to apply these teachings to methods for determining more than two types of target analytes, for example, three types, or four types, or more.

Generally, the plurality of capture objects are configured to be able to be spatially separated from each other. In some embodiment, the capture objects may be provided in a form such that the capture objects are capable of being spatially separated into a plurality of locations. For example, the plurality of capture objects may comprise a plurality of beads (which can be of any shape, e.g., sphere-like, disks, rings, cube-like, etc.), a dispersion or suspension of particulates (e.g., a plurality of particles in suspension in a fluid), nanotubes, or the like. In some embodiments, the plurality of capture objects is insoluble or substantially insoluble in the solvent(s) or solution(s) utilized in an assay. In some cases, the capture objects are non-porous solids or substantially non-porous solids (e.g., essentially free of pores); however, in some cases, the plurality of capture objects may be porous or substantially porous, hollow, partially hollow, etc. The plurality of capture objects may be non-absorbent, substantially non-absorbent, substantially absorbent, or absorbent. In some cases, the capture objects may comprise a magnetic material, which may facilitate certain aspect of an assay (e.g., washing step).

The plurality of capture objects may be of any suitable size or shape. Non-limiting examples of suitable shapes include spheres, cubes, ellipsoids, tubes, sheets, and the like. In certain embodiments, the average diameter (if substantially spherical) or average maximum cross-sectional dimension (for other shapes) of a capture object may be greater than about 0.1 um (micrometer), greater than about 1 um, greater than about 10 um, greater than about 100 um, greater than about 1 mm, or the like. In other embodiments, the average diameter of a capture object or the maximum dimension of a capture object in one dimension may be between about 0.1 um and about 100 um, between about 1 um and about 100 um, between about 10 um and about 100 um, between about 0.1 um and about 1 mm, between about 1 um and about 10 mm, between about 0.1 urn and about 10 urn, or the like. The “average diameter” or “average maximum cross-sectional dimension” of a plurality of capture objects, as used herein, is the arithmetic number average of the diameters/maximum cross-sectional dimensions of the capture objects. Those of ordinary skill in the art will be able to determine the average diameter/maximum cross-sectional dimension of a population of capture objects, for example, using laser light scattering, microscopy, sieve analysis, the Coulter effect, or other known techniques. For example, in some cases, a Coulter counter may be used to determine the average diameter of a plurality of beads.

In a particular embodiment, the objects comprise a plurality of beads. The beads may each comprise a plurality of targeting entities associated with at least a portion of each bead. In some embodiments, the beads are magnetic. The magnetic property of the beads may help in separating the beads from a solution and/or during washing step(s). Potentially suitable beads, including magnetic beads, are available from a number of commercial suppliers.

The capture objects may be fabricated from one or more suitable materials, for example, plastics or synthetic polymers (e.g., polyethylene, polypropylene, polystyrene, polyamide, polyurethane, phenolic polymers, or nitrocellulose etc.), naturally derived polymers (latex rubber, polysaccharides, polypeptides, etc.), composite materials, ceramics, silica or silica-based materials, carbon, metals or metal compounds (e.g., comprising gold, silver, steel, aluminum, copper, etc.), inorganic glasses, silica, and a variety of other suitable materials.

In some embodiments, each of the types of capture objects may be detectable. This property may be useful in embodiments where the fraction or percentage of capture objects associated with an analyte molecule is to be determined (e.g., when the total number of capture objects interrogated and detected is used to determine the fraction of capture objects associated with an analyte molecule). In a specific embodiment, the capture objects are detectable optically. For example, the location of a capture object may be detected by identifying the optical signature of the object by a conventional optical train and optical detection system. Depending on the optical signature and the operative wavelengths, optical filters designed for a particular wavelength may be employed for optical interrogation of the locations. For example, a capture object may be characterized as having an emission or absorption spectrum that can be exploited for detection so that capture objects may be interrogated to determine which spatial location contains a capture object. The properties of the emission spectrum (e.g., wavelength(s), intensity, etc.), may be selected such that the emission produced by the capture objects does not substantially alter and/or interfere with any other emission from components used in the assay (e.g., the emission of any labels used to determine the presence or absence of an analyte molecule). In some cases, dye molecules may be associated with a capture object.

Each type of capture object may be encoded to be distinguishable from each other (e.g., to facilitate differentiation upon detection) by including a differing detectable property. For example, each type of capture object may have a differing fluorescence emission, a spectral reflectivity, shape, a spectral absorption, or an FTIR emission or absorption. In a particular embodiment, each type of capture object may comprise one or more dye compounds (e.g., fluorescent dyes) but at varying concentration levels, such that each type of capture object has a distinctive signal (e.g., based on the intensity of the fluorescent emission). Generally, the non-targeting capture objects are distinguishable from the targeting capture objects, and each type of targeting capture object is distinguishable from the other types of targeting capture objects.

Those of ordinary skill in the art will be aware of methods and techniques for exposing a plurality of capture objects to a fluid sample or a solution derived from the fluid sample containing or suspected of containing at least one type of analyte molecule or particle for initial analyte capture. For example, the plurality of capture objects may be added (e.g., as a solid, as a solution) directly to a fluid sample. As another example, the fluid sample may be added to the plurality of capture objects (e.g., in solution, as a solid). In some instances, the solutions may be agitated (e.g., stirred, shaken, etc.).

The plurality of capture objects, subsequent to the exposing step (e.g., at least some capture objects are associated with at least one analyte molecule), may be exposed to one or more additional reagents, prior to spatially separating the plurality of capture objects (e.g., into a plurality of locations). For example, the capture objects may be exposed a plurality of binding ligands, at least some of which may associate with an immobilized analyte molecule. The capture objects may be exposed to more than one type of binding ligand (e.g., a first type of binding ligand and a second, third, etc. type of a binding ligand), as noted above. The association of a binding ligand with an immobilized analyte molecule may aid in the detection of the analyte molecules, as described herein. Additional details are described herein.

As described above, following immobilization of a plurality of analyte molecules with respect to the plurality of capture objects in the analyte capture step, at least a portion of the capture objects may be spatially separated into a plurality of locations, for example on a substrate. For example, each of capture objects of the portion of capture objects which are spatially separated may be positioned in and/or associated with a location (e.g., a spot, region, well, etc. on the surface and/or in the body of a substrate) that spatially distinct from the locations in which each of the other capture objects are located, such that the capture objects and locations can be individually resolved by an analytical detection system employed to address the locations. As an example, each of a portion of the capture objects may be spatially separated into an array of reaction vessels on a substrate, such that statistically only zero or one capture objects are located in at least some of the reaction vessels and in certain cases in essentially each reaction vessel. Each location may be individually addressable relative to the other locations. Additionally, in some embodiments, the locations may be arranged such that a plurality of locations may be addressed substantially simultaneously, as described herein, while still permitting the ability to resolve individual locations and capture objects. While exemplary embodiments for spatially separating a plurality of capture objects into a plurality of locations are described herein, numerous other methods may potentially be employed.

It should be understood, that while much of the discussion herein focusing on locations containing a single capture object, this is by no means limiting, and in some embodiments, more than one capture object may be contained at a single location. In such embodiments, the ratio of capture objects to analyte molecules may be such that following spatial separation of the plurality of capture objects into the plurality of locations, a statistically significant fraction of the locations contain no analyte molecules and a statistically significant fraction of locations contain at least one analyte molecule. That is, while a single location may contain a plurality of capture objects, in some cases, none of the capture objects are associated with any analyte molecules and only a single one of the capture objects in an addressed location is associated with at least one analyte molecule.

In embodiments wherein the plurality of locations comprise a plurality of reaction vessels, the plurality of capture objects may be spatially separated into the plurality of reaction vessels using any of a wide variety of techniques known to those of ordinary skill in the art. In some cases, the plurality of reaction vessels may be exposed to a solution containing the plurality of capture objects. In some embodiments, the plurality of reactions vessels may be exposed to a solution containing the plurality of capture objects using microfluidic techniques (e.g., see U.S. Publication No. 2011/0212848, by Duffy et al., filed Mar. 24, 2010; and International Publication No. WO2011/109364, by Duffy et al., filed Mar. 1, 2011, herein incorporated by reference). In some cases, time may elapsed following exposure of the reaction vessels to the solution to allow for the capture objects enter the wells (e.g., no applied force, gravity only). In some cases, force may be applied to the solution and/or capture objects, thereby aiding in the spatial separation of the capture objects from the fluid phase and/or the deposition of the capture objects in the vessels. For example, after application of an assay solution containing the capture objects to a substrate containing the reaction vessels, the substrate and solution may be centrifuged to assist in depositing the capture objects in the reaction vessels. In embodiments where the capture objects (e.g., beads) are magnetic, a magnet may be used to aid in containing the capture objects in the reaction vessels. In some cases, when the plurality of reaction vessels is formed on the end of a fiber optic bundle (or another planar surface), a material (e.g., tubing) may be placed around the edges of the surface of the array comprising the plurality of reaction vessel to form a container to hold the solution in place while the capture objects settle in the reaction vessels or are placed into the reaction vessels (e.g., while centrifuging). Following placement of the capture objects into at least some of the reaction vessels, the surrounding material may be removed and the surface of the array may be washed and/or swabbed to remove any excess solution/capture objects.

In some embodiments, the reaction vessels may all have approximately the same volume. In other embodiments, the reaction vessels may have differing volumes. The volume of each individual reaction vessel may be selected to be appropriate to facilitate any particular assay protocol. For example, in one set of embodiments where it is desirable to limit the number of capture objects used for analyte capture contained in each vessel to a small number, the volume of the reaction vessels may range from attoliters or smaller to nanoliters or larger depending upon the nature of the capture objects, the detection technique and equipment employed, the number and density of the wells on the substrate and the expected concentration of capture objects in the fluid applied to the substrate containing the wells. In one embodiment, the size of the reaction vessel may be selected such only a single capture object used for analyte capture can be fully contained within the reaction vessel (see, for example, U.S. Publication No. 2011/0212848, by Duffy et al., filed Mar. 24, 2010; and International Publication No. WO2011/109364, by Duffy et al., filed Mar. 1, 2011, herein incorporated by reference).

In accordance with one embodiment of the present invention, the reaction vessels may have a volume between about 1 femtoliter and about 1 picoliter, between about 1 femtoliters and about 100 femtoliters, between about 10 attoliters and about 100 picoliters, between about 1 picoliter and about 100 picoliters, between about 1 femtoliter and about 1 picoliter, or between about 30 femtoliters and about 60 femtoliters. In some cases, the reaction vessels have a volume of less than about 1 picoliter, less than about 500 femtoliters, less than about 100 femtoliters, less than about 50 femtoliters, or less than about 1 femtoliter. In some cases, the reaction vessels have a volume of about 10 femtoliters, about 20 femtoliters, about 30 femtoliters, about 40 femtoliters, about 50 femtoliters, about 60 femtoliters, about 70 femtoliters, about 80 femtoliters, about 90 femtoliters, or about 100 femtoliters.

The total number of locations and/or density of the locations employed in an assay (e.g., the number/density of reaction vessels in an array) can depend on the composition and end use of the array. For example, the number of reaction vessels employed may depend on the number of types of analyte molecule and/or binding ligand employed, the suspected concentration range of the assay, the method of detection, the size of the capture objects, the type of detection entity (e.g., free labeling agent in solution, precipitating labeling agent, etc.). Arrays containing from about 2 to many billions of reaction vessels (or total number of reaction vessels) can be made by utilizing a variety of techniques and materials. Increasing the number of reaction vessels in the array can be used to increase the dynamic range of an assay or to allow multiple samples or multiple types of analyte molecules to be assayed in parallel. The array may comprise between one thousand and one million reaction vessels per sample to be analyzed. In some cases, the array comprises greater than one million reaction vessels. In some embodiments, the array comprises between about 1,000 and about 50,000, between about 1,000 and about 1,000,000, between about 1,000 and about 10,000, between about 10,000 and about 100,000, between about 100,000 and about 1,000,000, between about 100,000 and about 500,000, between about 1,000 and about 100,000, between about 50,000 and about 100,000, between about 20,000 and about 80,000, between about 30,000 and about 70,000, between about 40,000 and about 60,000 reaction vessels. In some embodiments, the array comprises about 10,000, about 20,000, about 50,000, about 100,000, about 150,000, about 200,000, about 300,000, about 500,000, about 1,000,000, or more, reaction vessels.

The array of reaction vessels may be arranged on a substantially planar surface or in a non-planar three-dimensional arrangement. The reaction vessels may be arrayed in a regular pattern or may be randomly distributed. In a specific embodiment, the array is a regular pattern of sites on a substantially planar surface permitting the sites to be addressed in the X-Y coordinate plane.

The plurality of locations may be formed using any suitable technique and/or formed from any suitable material. In some embodiments, the plurality of locations comprises a plurality of reaction vessels/wells on a substrate. The reactions vessels, in certain embodiments, may be configured to receive and contain only a single capture object. In some embodiments, the reaction vessels are formed in a solid material. As will be appreciated by those in the art, the number of potentially suitable materials in which the reaction vessels can be formed is very large, and includes, but is not limited to, glass (including modified and/or functionalized glass), plastics (including acrylics, polystyrene and copolymers of styrene and other materials, polypropylene, polyethylene, polybutylene, polyurethanes, cyclic olefin copolymer (COC), cyclic olefin polymer (COP), Teflon®, polysaccharides, nylon or nitrocellulose, etc.), elastomers (such as poly(dimethyl siloxane) and poly urethanes), composite materials, ceramics, silica or silica-based materials (including silicon and modified silicon), carbon, metals, optical fiber bundles, or the like. In general, the substrate material may be selected to allow for optical detection without appreciable autofluorescence. In certain embodiments, the reaction vessels may be formed in a flexible material. A reaction vessel in a surface (e.g., substrate or sealing component) may be formed using a variety of techniques known in the art, including, but not limited to, photolithography, stamping techniques, molding techniques, etching techniques, or the like. As will be appreciated by those of the ordinary skill in the art, the technique used can depend on the composition and shape of the supporting material and the size and number of reaction vessels.

In some embodiments, the plurality of analyte molecules may be spatially separated into a plurality of locations such that at least some of the locations contain at least one analyte molecule and a statistically significant fraction of the locations contain no analyte molecules. A statistically significant fraction of reaction vessels that contain at least one analyte molecule (or no analyte molecules) will typically be able to be reproducibly detected and quantified using a particular system of detection and will typically be above the background noise (e.g., non-specific binding) that is determined when carrying out the assay with a sample that does not contain any analyte molecules, divided by the total number of locations addressed. A “statistically significant fraction” as used herein for the present embodiments, may be estimated according to the Equation 1:

$${P}_{μ} ( ν ) = {e}^{- μ} ( \frac{{μ}^{ν}}{ν !} ) \qquad ( Eq . 1 )$$

wherein n is the number of determined events for a selected category of events. That is, a statistically significant fraction occurs when the number of events n is greater than three times square root of the number of events. For example, to determine a statistically significant fraction of the locations which contain an analyte molecule or particle, n is the number of locations which contain an analyte molecule. As another example, to determine a statistically significant fraction of the capture objects associated with a single analyte molecule, n is the number of capture objects associated with a single analyte molecule.

In some embodiments, the statistically significant fraction of locations that contain at least one analyte molecule associated with a targeting capture object to the total number of targeting capture objects is less than about 1:2, less than about 1:3, less than about 1:4, less than about 2:5, less than about 1:5, less than about 1:10, less than about 1:20, less than about 1:100, less than about 1:200, or less than about 1:500. Therefore, in such embodiments, the fraction of targeting capture objects not containing any analyte molecules to the total number of targeting capture objects is at least about 1:100, about 1:50, about 1:20, about 1:10, about 1:5, about 1:4, about 1:3, about 1:2, about 1:1, about 2:1, about 3:1, about 4:1, about 5:1, about 10:1, about 20:1, about 50:1, about 100:1, or greater.

Non-limiting methods and techniques for spatially separating a plurality of capture objects into a plurality of locations is described in, for example, U.S. Publication No. 2011/0212848 by Duffy et al., filed Mar. 24, 2010; International Publication No. WO2011/109364 by Duffy et al., filed Mar. 1, 2011; U.S. Publication No. 2007/0259448, by Walt et al., filed Feb. 16, 2007; U.S. Publication No. 2007/0259385, by Walt et al., filed Feb. 16, 2007; U.S. Publication No. 2007/0259381, by Walt et al., filed Feb. 16, 2007; International Publication No. WO2009/029073, by Walt et al., filed Aug. 20, 2007; and International Publication No. WO2010/039179, by Duffy et al., filed Sep. 9, 2009, each herein incorporated by reference.

It should be understood that while many of the embodiments described herein utilize locations comprising reaction vessels, this is by no means limiting, and other types of locations may be utilized. In addition, the locations need not be necessarily detectable substantially simultaneously. For example, in some embodiments, the capture objects subjected to the exposing step may be analyzed using methods and techniques wherein the capture objects are detected sequentially or partially sequentially, for example, using flow cytometry, microfluidics, etc. These and other similar methods and systems will be known to those of ordinary skill in the art. In some cases, a patterned substantially planar surface may be employed, wherein the patterned areas form a plurality of locations. In some cases, the patterned areas may comprise substantially hydrophilic surfaces which are substantially surrounded by substantially hydrophobic surfaces. A plurality of capture objects (e.g., beads) may be substantially surround by a substantially hydrophilic medium (e.g., comprising water), and the beads may be exposed to the pattern surface such that the beads associate in the patterned areas (e.g., the locations), thereby spatially separating the plurality of beads. For example, in one such embodiment, a substrate may be or include a gel or other material able to provide a sufficient barrier to mass transport (e.g., convective and/or diffusional barrier) to prevent capture objects used for analyte capture and/or precursor labeling agent and/or labeling agent from moving from one location on or in the material to another location so as to cause interference or cross-talk between spatial locations containing different capture objects during the time frame required to address the locations and complete the assay. For example, in one embodiment, a plurality of capture objects is spatially separated by dispersing the capture objects on and/or in a hydrogel material. As still yet another embodiment, the capture objects may be confined in one or more capillaries. In some cases, the plurality of capture objects may be absorbed or localized on a porous or fibrous substrate, for example, filter paper. In some embodiments, the capture objects may be spatially separated on a uniform surface (e.g., a planar surface), and the capture objects may be detected using precursor labeling agents which are converted to substantially insoluble or precipitating labeling agents that remain localized at or near the location of where the corresponding capture object is localized. As another non-limiting embodiment, in some cases, the capture objects may be spatially separated into a plurality of droplets (e.g., using microfluidic techniques).

Following spatial separation of at least a portion of the capture objects into a plurality of locations, at least a portion of the locations may be addressed to determine a measure of the number and/or a measure of the concentration of a target analyte in the fluid sample. In some embodiments, at least a portion of the locations may be addressed and a measure indicative of the number/percentage/fraction of the locations containing at least one target analyte molecule may be made. In some cases, based upon the number/percentage/fraction, a measure of the concentration of the target analyte in the fluid sample may be determined. The measure of the concentration of target analyte molecules in the fluid sample may be determined by a digital analysis method/system optionally employing Poisson distribution adjustment and/or based at least in part on a measured intensity of a signal, as will be known to those of ordinary skill in the art. In some cases, the assay methods and/or systems may be automated. In some embodiments, the capture objects may be detected substantially simultaneously. However, in other embodiments, the capture objects may be detected sequentially.

In some embodiments, the locations addressed may be locations that contain at a certain type of analyte molecule and/or capture object. In other embodiments, the locations addressed may be locations which contain at least one targeting capture object (e.g., either associated with or not associated with any analyte molecules), and thus, in these embodiments, the percentage of locations containing at least one analyte molecule is also the percentage of capture objects associated with at least one analyte molecule (e.g., the percentage “active” beads). For example, in some embodiments, a measure of the concentration of analyte molecules or particles in the fluid sample may be determined based at least in part on the number/percentage of targeting capture objects associated with at least one analyte molecule or particle.

In certain embodiments, a measure of the concentration of a target analyte molecules in the fluid sample may be determined based on the information received when addressing the locations (e.g., using the information received from the imaging system and/or processed using a computer implemented control system). In some embodiments, the number of locations which contain a targeting capture object are determined. In some embodiments, the number of locations which contain a targeting capture object and an analyte molecule are determined. In some cases, a measure of the concentration may be based at least in part on the number of locations determined to contain a targeting capture object that is or was associated with the corresponding target analyte. In some cases, a measure of the concentration may be based at least in part on the number of locations determined to contain a targeting capture object that is or was associated with the corresponding target analyte. The number of locations which contain non-targeting capture object may or may not be determined. In other cases and/or under differing conditions, a measure of the concentration may be based at least in part on an intensity level of at least one signal indicative of the presence of a plurality of target analyte molecule and/or capture objects associated with a target analyte molecule at one or more of the addressed locations. In some embodiments, the locations may be interrogated optically.

In some embodiments, a measure of the concentration of a first type of target analyte in the fluid sample may be determined at least in part using a calibration curve developed using samples containing known concentrations of the first type of target analyte molecules. In some cases, a measure of the concentration of first type of target analyte in the fluid sample may be determined at least in part by comparison of a measured parameter to a calibration standard. In some cases, a calibration curve may be prepared, wherein the total measured signal is determined for a plurality of samples comprising the first type of target analyte at a known concentration using a substantially similar assay format. For example, the number and/or fraction of locations that comprise a first type of target analyte associated with a target capture object, or alternatively, the average intensity of the capture objects in the array, may be compared to a calibration curve to determine a measure of the concentration of the first type of target analyte in the fluid sample. The calibration curve may be produced by completing the assay with a plurality of standardized samples of known concentration under similar conditions used to analyze test samples with unknown concentrations. A calibration curve may relate the number and/or fraction of the locations determined to contain a target capture object associated with first type of target analyte with a known concentration of the first type of target analyte. The assay may then be completed on a sample containing the first type of target analyte in an unknown concentration, and number/fraction of locations containing a target capture object associated with a first type of target analyte may be compared to the calibration curve, (or a mathematical equation fitting same) to determine a measure of the concentration of the first type of target analyte in the fluid sample. Similar analysis/calibration may be utilized for additional types of target analytes.

In some embodiments, the non-target capture objects may be used for a number of other purposes alternatively or in addition to those described above including, but not limited to, identification of the orientation of the plurality of locations (e.g., in the case where the plurality of locations is formed as an array of reaction sites, reaction vessels, etc.), to help determine the quality of the assay, and/or to help calibrate the detection system (e.g., optical interrogation system), as described below. It should be understood, when more than one type of non-targeting capture object is utilized, each type of capture object may or might not be utilized for any of these other purposes. For example, each type of capture object may be utilized for the same purpose, or each type of capture object may be used for different purposes, and/or at least one type of capture object may not be used for any of the purposes specifically described. For example, a first type of non-target capture object may be used to determine quality of the assay and a second type of non-target capture object may be used to act as a location marker.

In some cases, the non-targeting capture objects may be used to identify the orientation of the plurality of locations (e.g., reaction vessels, sites, etc.) on an array (e.g., function as location marker(s) for an array). For example, a non-targeting capture object may be randomly or specifically distributed on an array, and may provide one or more reference locations for determining the orientation/position of the array. Such a feature may be useful when comparing multiple images of a portion of the array at different time intervals. That is, the positions of non-targeting capture objects in the array may be used to register the images. In some cases, the non-targeting capture objects may be use to provide reference locations in embodiments where a plurality of images of small overlapping regions are being combined to form a larger image.

In some embodiments, the non-targeting capture objects may be used to provide information regarding the quality of the assay. For example, if a location is found to contain a non-targeting capture object comprising an enzymatic component but no labeling agent is present (e.g., the product of which would be present upon exposure of a non-targeting capture object comprising an enzymatic component to a precursor labeling agent), this gives an indication that some aspect of the assay may not be functioning properly. For example, the quality of the reagents may be compromised (e.g., concentration of precursor labeling agent is too low, decomposition of the precursor labeling agent has occurred, etc.), and/or perhaps not all of the locations were exposed to the precursor labeling agent.

In some embodiments, the non-targeting capture objects may be used to calibrate the detection system. For example, the non-targeting capture objects may output an optical signal which may be used to calibrate an optical detection system. In some embodiments, the non-targeting capture objects can be characterized and doped with a particular detectable characteristic (e.g., fluorescence, color, absorbance, etc.) which can act as a quality control check for the detection system performance.

In some cases, the non-targeting capture objects may be used to standardize and/or normalize the assay and/or system to account for variations of the performance and/or characteristics of different system components in different assays, over the course of time, etc. (e.g., detection system, arrays, reagents, local environment, etc.), between different portions of an array used in a test, and/or between two different arrays. For example, experimental set-up, parameters, and/or variations may lead to changes the intensity of a signal (e.g., fluorescence signal) produced from the beads in a single array at different time points, or between the beads in at least two arrays at simultaneous or different time points. In addition, in a single array, different portions of the array may produce different background signals. Such variations may lead to changes in calibration signals (e.g., differences in the determined average bead signal) between arrays, portions of and array or at multiple times, which can lead to inaccurate determinations in some cases. Non-limiting examples of parameters that may cause variation include labeling agent concentration, temperature, focus, intensity of detection light, depth and/or size of the locations in an array, reduction in activity of reagents (e.g., enzyme label), etc. To account for the effects of some or all of such variations, in some embodiments, a plurality of non-targeting capture objects may be utilized, wherein each type of capture object may be used to standardize and/or normalize the assay with respect to one or more of the parameters.

In some embodiments, the signals from the non-targeting capture objects may be used to normalize the interrogation values between different arrays, or between different areas of a single array. For example, because the signals from the non-targeting capture objects should be approximately equal between arrays and/or about different areas of a single array, the non-targeting capture object signals may be normalized to an appropriate value and the signals of the targeting capture objects associated with an analyte molecule may be adjusted accordingly).

In one embodiment, the non-targeting capture objects may comprise a positive control and include an enzymatic component. A precursor labeling agent may be converted to a labeling agent upon exposure to the enzymatic component. In some cases, the enzymatic component may be the same as the enzymatic component being used to detect the analyte molecules in a fluid sample (e.g., utilized in another component of the assay, for example, an enzymatic component associated with a binding ligand, an analyte molecule, etc.). In such embodiments, the non-targeting capture object may be distinguishable from the targeting capture objects such that the reaction vessels having a positive signal may be analyzed to determine whether the reaction vessel comprises a non-targeting capture object (e.g., having a first detectable signal) or a targeting capture object (e.g., having a second detectable signal distinguishable from the first detectable signal). In other cases, the enzymatic component may be different than an enzymatic component being used to detect the analyte molecules in a fluid sample (e.g., utilized in another component of the assay, for example, an enzymatic component associated with a binding ligand, an analyte molecule, etc.). In such an embodiment, the non-targeting capture object may or may not be distinguishable from the targeting capture objects. Both a first type and a second type of precursor labeling agent may be provided to the reaction vessels, and the first type of precursor labeling agent may be converted to a first type of labeling agent upon exposure to the enzymatic component associated with the non-targeting capture beads and the second type of precursor labeling agent may be converted to a second type of labeling agent upon exposure to the other enzymatic component (e.g., associated with the binding ligand/analyte molecule/etc.). The reaction vessels containing the first type of labeling agent correspond to the reaction vessels containing a non-targeting capture object and reaction vessels containing a second type of labeling agent correspond to the reaction vessels which contain a binding ligand/analyte molecule/etc. The plurality of locations containing a non-targeting capture bead may be analyzed, for example to determine the effectiveness of the enzymatic conversion reaction. In such cases, the targeting and non-targeting capture objects may or might not be distinguishable from each other.

In some embodiments, at least one type of non-targeting capture object may include at least one binding surface having specific affinity for a second type of analyte molecule, wherein the concentration of the second type of analyte molecule need not be determined but which is detected for other reasons (e.g., to calibrate the system, etc.), as described in more detail herein. In some embodiments, the second type of capture object may be a binding ligand (e.g., used in the detection of the first type of analyte molecule or particle) or a non-target analyte molecule. In some embodiments, a non-target capture object may capture a second type of analyte molecule, wherein the second type of analyte molecule may be detected either directly or indirectly. In some embodiments, the second type of analyte molecule may include an enzymatic component and/or may be exposed to a binding ligand which comprises an enzymatic component or associates with an enzymatic component. Similar to above, a precursor labeling agent may be converted to a labeling agent upon exposure to the enzymatic component. In some cases, the enzymatic component may be the same as the enzymatic component being used to detect the target analyte molecules in a fluid sample. In some embodiments, the second type of analyte molecule may be directly detectable.

In some embodiments, a non-targeting capture object itself may include a surface having a plurality of targeting moieties which can associate with and/or capture the second type of analyte molecule. In some embodiments, the second type of analyte molecule comprises an enzymatic component. For example, the non-targeting capture object may comprises a plurality of biotin molecules, wherein the biotin molecules may be used to capture streptavidin-enzyme conjugates. As another example, the non-targeting capture object may comprises a plurality of at least one type of antibody to beta-galactosidase, wherein the antibodies to beta-galactosidase may be used to capture streptavidin-beta-galactosidase. As another example, the non-targeting capture objects may include a surface which has specific affinity for the binding ligands used in the assay to detect a target analyte. For example, if the assay employs a binding ligand comprising a detection antibody directly conjugated to an enzyme for detection, the non-targeting capture object may include a surface having specific affinity for the detection antibody or the conjugated enzyme (e.g., anti-enzyme antibodies such as anti-beta-galactosidase) or antibodies that bind to the host of the detection antibody (e.g., anti-rabbit antibodies for detection antibodies raised in rabbits).

As will be understood by those of ordinary skill in the art, the amount/quantity of targeting moieties on the surface of the non-targeting capture objects may be selected to allow the desired amount of the second type of analyte molecule to associate with the non-targeting capture object. For example, one of ordinary skill in the art will be able to carry out screening tests to determine an appropriate amount/quantity of biotin or other targeting moieties (e.g., an antibody to beta-galactosidase) to be present on the surface of each of the non-targeting capture objects such that approximately one or zero enzymatic components or other detectable components associate with each non-targeting capture object. In some cases, the amount/quantity of targeting moieties on the surface of reach non-target capture object is selected so that a statistically significant fraction of the non-targeting capture objects associate with a single detectable component (e.g., enzymatic label), while minimizing Poisson noise. In some embodiments, between about 1% and about 20%, or between about 5% and about 20%, or between about 10% and about 20% of the non-target capture objects associate with a single detectable component (e.g., a second type of binding ligand, an enzymatic component, etc.).

In some embodiments, the non-target capture objects may not include any specific binding surfaces having specific affinity for any target analyte molecules or non-target analyte molecules, however, such surfaces may non-specifically bind to a target analyte or non-target analyte molecule. That is, non-specific binding (NSB) may be used to capture certain molecules for detection. For example, as will be known to those of ordinary skill in the art, certain types of proteins are known to be “sticky” (e.g., fibrinogen) wherein the proteins have a tendency to non-specifically bind a number of components. As another example, hydrophobic proteins may be associated with the surface of the non-targeting capture objects, wherein the hydrophobic proteins aid in non-specific binding of a detectable component. Similar to above, those of ordinary skill in the art will be able to vary the amount/quantity of non-specific targeting moieties (e.g., sticky protein) present on the surface of a non-target capture object such that approximately one or zero second type of analyte molecules associate with each non-targeting capture objection. In some cases, the amount/quantity of non-specific targeting moieties on the surface of each non-target capture object is selected so that a statistically significant fraction of the non-targeting capture objects associate with a second type of target analyte, while minimizing Poisson noise. In some embodiments, between about 1% and about 20%, or between about 5% and about 20%, or between about 10% and about 20% of the non-target capture objects associate with a second type of analyte molecule via non-specific binding.

As described above, in some embodiments, a method for determining a measure of the concentration of a first type of analyte molecules or particles in a fluid sample comprises exposing a plurality of capture objects to a solution containing or suspected of containing a first type of analyte molecules or particles, wherein the capture objects comprise a first type of capture object and a second type of capture object, and exposing the plurality of capture objects to a second type of analyte molecules or particles. Each of the first type of capture object includes a binding surface having specific binding affinity for the first type of analyte molecule or particle and each of the second type of capture objects do not include any binding surfaces having specific binding affinity for the first type of analyte molecules or particles contained in or suspected to be contained in the solution and include at least one binding surface having some affinity for the second type of analyte molecule or particle. In such embodiments, at least some of the first type of capture objects associate with at least one analyte molecule or particle and at least some of the first type of capture objects do not associate with any analyte molecules or particles and a statistically significant fraction of the second type of capture objects associate with either zero or one of the second type of analyte molecules or particles. The ratio of the number of first type of capture objects to the total number of capture objects may be as described herein (e.g., between 1:1.2 and 1:100). At least a portion of the plurality of capture objects subjected to the exposing step may be spatially separated into a plurality of separate locations, wherein at least some of the locations are addressed to determine the number of locations containing a first type of capture object, the number of locations determined to contain a first type of capture object that also contain a first type of analyte molecule or particle, and the number of locations which contain a second type of capture object and a second type of analyte molecule or particle (e.g. a binding ligand.) The average intensity of the locations containing a second type of capture object and a second type of analyte molecule or particle may be determined, and a measure of the concentration of the first type of analyte molecules or particles in the fluid sample may be determined based at least in part on the average intensity of the locations containing a second type of capture object and a second type of analyte molecule or particle.

As a specific example, in some embodiments, the non-targeting capture objects may be used to determine the value, I_{single}. The term I_{single} is used herein to refer to the average intensity of at least a portion of the locations containing a single analyte molecule. In some embodiments, I_{single} is utilized wherein the analyte molecules are detected using enzymatic components. In some embodiments, the value I_{single_} is used to help normalize variation in the assay conditions which may affect the determination of the measure of the concentration of the target analyte molecule(s). In some embodiments, the measure of the concentration of a target analyte is determined, in part, via determination of the average-enzyme-per-bead (AEB) for each type of analyte molecule, wherein the AEB for a given analyte molecule is determined by the equation fₒₙ×I_{bead}/I_{single}, wherein fₒₙ is the fraction of “on” beads for a given type of target analyte molecule, I_{bead} is the average fluorescence intensity value of the active beads, and I_{single} is the average fluorescence intensity generated by a single enzyme. In some embodiments, the I_{single} previously used was based on an average I_{single_} determined in a large number of assays. Such calculations may give rise to errors because variations in the assay (e.g., as described above, including temperature) can vary enzyme activity and thus, I_{single}. Accordingly, using an averaged I_{single} over many assays might not be representative of the value at the time of the present sample measurement. Additional details regarding the determination of the concentration of analyte molecules using AEB are described in U.S. Publication No. 2011/0212537, by Duffy et al., filed Mar. 24, 2010; and U.S. Publication No. 2011/0245097 by Rissin et al., filed Mar. 1, 2011, each herein incorporated by reference.

Accordingly, in some embodiments, instead of using an average I_{single_} determined in a large number of assays, I_{single} may be determined uniquely for each assay utilizing the non-target capture objects (e.g., wherein I_{single} is the average intensity of the locations determined to contain a second type of capture object and a second type of analyte molecule). For example, as described above, the non-target capture objects may associate with either zero or one second type of analyte molecule and I_{single} can be determined for the assay by analyzing the data and determining the average intensity for at least a portion of the locations containing a non-target capture object and a second type of analyte molecule. That is, I_{single} for the assay is determined via analyzing the non-target capture objects which associate with an second type of analyte molecule and averaging the signal detected for those non-target capture objects to provide I_{single}.

In some embodiments, at least one type of non-targeting capture object comprises one or more targeting moieties which have some binding affinity (e.g., specific affinity, or non-specific affinity as described above) for at least one type of binding ligand (e.g., comprising a enzymatic component) which is used to detect the target analyte. For example, in some embodiments, the target analyte molecule may be detected via exposure to a first type of binding ligand comprising biotin, and a second type of binding ligand comprising streptavidin-β-galactosidase, wherein the first binding ligand associates with the target analyte and the second type of binding ligand associates with the first type of binding ligand. In such embodiments, the non-target capture object may comprise a plurality of biotin molecules associated with at least one surface of the non-target capture object, wherein the second type of binding ligand may associate with at least one biotin. In some embodiment, this approach offers a “within array” calibration to account for the many of the variables that can affect I_{single} as described above.

In some embodiments, a plurality of locations may be addressed and/or a plurality of capture objects and/or species/molecules/particles of interest may be detected substantially simultaneously. “Substantially simultaneously” when used in this context, refers to addressing/detection of the locations/capture objects/species/molecules/particles of interest at approximately the same time such that the time periods during which at least two locations/capture objects/species/molecules/particles of interest are addressed/detected overlap, as opposed to being sequentially addressed/detected, where they would not. Simultaneous addressing/detection can be accomplished by using various techniques, including optical techniques (e.g., CCD detector, scanning). Spatially separating capture objects/species/molecules/particles into a plurality of discrete, resolvable locations, according to some embodiments facilitates substantially simultaneous detection by allowing multiple locations to be addressed substantially simultaneously. For example, for embodiments where individual species/molecules/particles are associated with capture objects that are spatially separated with respect to the other capture objects into a plurality of discrete, separately resolvable locations during detection, substantially simultaneously addressing the plurality of discrete, separately resolvable locations permits individual capture objects, and thus individual species/molecules/particles (e.g., analyte molecules) to be resolved. For example, in certain embodiments, individual molecules/particles of a plurality of molecules/particles are partitioned across a plurality of reaction vessels such that each reaction vessel contains zero or only one species/molecule/particle. In some cases, between about 0.1% and about 50%, or between about 0.1% and about 40%, or between about 0.1% and about 30%, or between about 0.1% and about 20%, or between about 0.1% and about 10%, or between about 0.5% and about 10%, or between about 1% and about 10% of all species/molecules/particles are spatially separated with respect to other species/molecules/particles during detection. A plurality of species/molecules/particles may be detected substantially simultaneously within a time period of less than about 1 second, less than about 500 milliseconds, less than about 100 milliseconds, less than about 50 milliseconds, less than about 10 milliseconds, less than about 1 millisecond, less than about 500 microseconds, less than about 100 microseconds, less than about 50 microseconds, less than about 10 microseconds, less than about 1 microsecond, less than about 0.5 microseconds, less than about 0.1 microseconds, or less than about 0.01 microseconds, less than about 0.001 microseconds, or less. In some embodiments, the plurality of species/molecules/particles may be detected substantially simultaneously within a time period of between about 100 microseconds and about 0.001 microseconds, between about 10 microseconds and about 0.01 microseconds, or less.

In some embodiments, the locations are optically interrogated. The locations exhibiting changes in their optical signature may be identified by a conventional optical train and optical detection system. Depending on the detected species (e.g., type of fluorescence entity, etc.) and the operative wavelengths, optical filters designed for a particular wavelength may be employed for optical interrogation of the locations. In embodiments where optical interrogation is used, the system may comprise more than one light source and/or a plurality of filters to adjust the wavelength and/or intensity of the light source. In some embodiments, the optical signal from a plurality of locations is captured using a CCD camera.

In some embodiments, the analyte molecules (e.g., optionally associated with a capture objects) may be exposed to at least one reagent. In some cases, the reagent may comprise a plurality of binding ligands which have an affinity for at least one type of analyte molecule (or particle). A “binding ligand,” is any molecule, particle, or the like which specifically binds to or otherwise specifically associates with an analyte molecule to aid in the detection of the analyte molecule. Certain binding ligands can comprise an entity that is able to facilitate detection, either directly (e.g., via a detectable moiety) or indirectly. A component of a binding ligand may be adapted to be directly detected in embodiments where the component comprises a measurable property (e.g., a fluorescence emission, a color, etc.). A component of a binding ligand may facilitate indirect detection, for example, by converting a precursor labeling agent into a labeling agent (e.g., an agent that is detected in an assay). Accordingly, another exemplary reagent is a precursor labeling agent. A “precursor labeling agent” is any molecule, particle, or the like, that can be converted to a labeling agent upon exposure to a suitable converting agent (e.g., an enzymatic component). A “labeling agent” is any molecule, particle, or the like, that facilitates detection, by acting as the detected entity, using a chosen detection technique. In some embodiments, the binding ligand may comprise an enzymatic component (e.g., horseradish peroxidase, beta-galactosidase, alkaline phosphatase, etc.). A first type of binding ligand may or may not be used in conjunction with additional binding ligands (e.g., second type, etc.).

In some embodiments, the analyte molecules may be directly detected or indirectly detected. In the case of direct detection, the analyte molecule may comprise a molecule or moiety that may be directly interrogated and/or detected (e.g., a fluorescent entity). In the case of indirect detection, an additional component is used for determining the presence of the analyte molecule. In some cases, the analyte molecules may be composed to a precursor labeling agent (e.g., enzymatic substrate) and the enzymatic substrate may be converted to a detectable product (e.g., fluorescent molecule) upon exposure to an analyte molecule. In some cases, the plurality analyte molecules may be exposed to at least one additional reaction component prior to, concurrent with, and/or following spatially separating at least some of the analyte molecules into a plurality of locations. In some cases, a plurality of capture objects at least some associated with at least one analyte molecule may be exposed to a plurality of binding ligands. In certain embodiments, a binding ligand may be adapted to be directly detected (e.g., the binding ligand comprises a detectable molecule or moiety) or may be adapted to be indirectly detected (e.g., including a component that can convert a precursor labeling agent into a labeling agent), as discussed more below. More than one type of binding may be employed in any given assay method, for example, a first type of binding ligand and a second type of binding ligand. In one example, the first type of binding ligand is able to associate with a first type of analyte molecule and the second type of binding ligand is able to associate with the first binding ligand. In another example, both a first type of binding ligand and a second type of binding ligand may associate with the same or different epitopes of a single analyte molecule, as described herein.

In some embodiments, at least one binding ligand comprises an enzymatic component. In some embodiments, the analyte molecule may comprise an enzymatic component. The enzymatic component may convert a precursor labeling agent (e.g., an enzymatic substrate) into a labeling agent (e.g., a detectable product). A measure of the concentration of analyte molecules in the fluid sample can then be determined based at least in part by determining the number of locations containing a labeling agent (e.g., by relating the number of locations containing a labeling agent to the number of locations containing an analyte molecule (or number of capture objects associated with at least one analyte molecule to total number of capture objects)). Non-limiting examples of enzymes or enzymatic components include horseradish peroxidase, beta-galactosidase, and alkaline phosphatase. Other non-limiting examples of systems or methods for detection include embodiments where nucleic acid precursors are replicated into multiple copies or converted to a nucleic acid that can be detected readily, such as the polymerase chain reaction (PCR), rolling circle amplification (RCA), ligation, Loop-Mediated Isothermal Amplification (LAMP), etc. Such systems and methods will be known to those of ordinary skill in the art, for example, as described in “DNA Amplification: Current Technologies and Applications,” Vadim Demidov et al., 2004.

In certain embodiments, solubilized, or suspended precursor labeling agents may be employed, wherein the precursor labeling agents are converted to labeling agents which are insoluble in the liquid and/or which become immobilized within/near the location (e.g., within the reaction vessel in which the labeling agent is formed). Such precursor labeling agents and labeling agents and their use is described in commonly owned U.S. Publication No. 2010/0075862, by Duffy et al., filed Sep. 23, 2008, incorporated herein by reference.

In some embodiments, when the plurality of locations comprise a plurality of reaction vessels, the plurality of reaction vessels may be sealed (e.g., after the introduction of the target analyte molecules, binding ligands, etc.), for example, through the mating of the second substrate and a sealing component. Non-limiting examples films that a sealing component may comprise include solid films (e.g., of a compliant material), fluid films (e.g., of fluids substantially immiscible with sample fluid contained in the assay sites), or the like. The sealing of the reaction vessels may be such that the contents of each reaction vessel cannot escape the reaction vessel during the remainder of the assay. In some embodiments, the sealing component may be a fluid. The fluid comprising the sealing component is advantageously substantially immiscible with the fluid contained in the assay sites. As used herein, a “fluid” is given its ordinary meaning, i.e., a liquid or a gas. The fluid may have any suitable viscosity that permits flow. If two or more fluids are present, the fluids may each be substantially miscible or substantially immiscible. In some cases, the fluid(s) comprising the sealing component can miscible or partially miscible with the assay sample fluid at equilibrium, but may be selected to be substantially immiscible with the assay sample fluid within the time frame of the assay or interaction. Those of ordinary skill in the art can select suitable sealing fluids, such as fluids substantially immiscible with sample fluids, using contact angle measurements or the like, to carry out the techniques of the invention. In some cases, the sample fluid and/or rinsing fluid and/or reagent fluid is an aqueous solution and the sealing component comprises a non-aqueous fluid. Non-limiting examples of potentially suitable non-aqueous fluids include fluorous liquids, oils (e.g., mineral oils, fluorinated oils), ferrofluids, non-aqueous polymer solutions (e.g., thickeners), and the like. In other cases, the sample fluid and/or rinsing fluid and/or reagent fluid is a non-aqueous solution and the sealing component comprising an aqueous fluid. In some cases, the sample fluid is a hydrogel whose viscosity changes upon temperature or other physicochemical triggers.

During the method, one or more wash steps may be carried out using techniques known to those of ordinary skill in the art. A wash step may aid in the removal of any unbound molecules from the solution. A wash step may be performed using any suitable technique known to those of ordinary skill in the art, for example, by incubation of the objects with a wash solution followed by removal of the solution (e.g., in embodiments where small objects are employed such as beads, by centrifuging the solution comprising the objects and decanting off the liquid, or by using filtration techniques). In embodiments where the object is magnetic, the object may be isolated from the bulk solution with aid of a magnet.

In some embodiments, the optical signal may be captured using a CCD camera. Other non-limiting examples of camera imaging types that can be used to capture images include charge injection devices (CIDs), complementary metal oxide semiconductors (CMOSs) devices, scientific CMOS (sCMOS) devices, and time delay integration (TDI) devices, as will be known to those of ordinary skill in the art. The camera may be obtained from a commercial source. CIDs are solid state, two dimensional multi pixel imaging devices similar to CCDs, but differ in how the image is captured and read. For examples of CIDs, see U.S. Pat. Nos. 3,521,244 and 4,016,550. CMOS devices are also two dimensional, solid state imaging devices but differ from standard CCD arrays in how the charge is collected and read out. The pixels are built into a semiconductor technology platform that manufactures CMOS transistors thus allowing a significant gain in signal from substantial readout electronics and significant correction electronics built onto the device. For example, see U.S. Pat. No. 5,883,830. CMOS devices comprise CMOS imaging technology with certain technological improvements that allows excellent sensitivity and dynamic range. TDI devices employ a CCD device that allows columns of pixels to be shifted into and adjacent column and allowed to continue gathering light. This type of device is typically used in such a manner that the shifting of the column of pixels is synchronous with the motion of the image being gathered such that a moving image can be integrated for a significant amount of time and is not blurred by the relative motion of the image on the camera. In some embodiments, a scanning mirror system coupled with a photodiode or photomultiplier tube (PMT) could be used to for imaging.

The capture objects described herein may find use in a variety of applications. That is, wherein the application makes use of a plurality of types of objects, wherein each type of object is uniquely identifiable (e.g., via association with a unique type of reporter molecule or unique of reporter molecule amount) and uniquely targeted (e.g., via association of a unique target moiety, each unique targeting moiety being associated with a unique type or amount of reporter molecule). In some cases, the objects may comprise a plurality of beads, and the objects may be employed in the methods and systems described in those described in U.S. Publication No. 2007/0259448, by Walt et al., filed Feb. 16, 2007; U.S. Publication No. 2007/0259385, by Walt et al., filed Feb. 16, 2007; U.S. Publication No. 2007/0259381, by Walt et al., filed Feb. 16, 2007; International Publication No. WO2009/029073, by Walt et al., filed Aug. 30, 2007; U.S. Publication No. 2010/0075862, by Duffy et al., filed Sep. 23, 2008; U.S. Publication No. 2010/0075407, by Duffy et al., filed Sep. 23, 2008; U.S. Publication No. 2010/0075439, by Duffy et al., filed Sep. 23, 2008; U.S. Publication No. 2010/0075355, by Duffy et al., filed Sep. 23, 2008; U.S. Publication No. 2011/0212848, by Duffy et al., filed Mar. 24, 2010; U.S. Publication No. 2011/0212462, by Duffy et al., filed Mar. 24, 2010; U.S. Publication No. 2011/0212537, by Duffy et al., filed Mar. 24, 2010; U.S. Publication No. 2012/0196774 by Fournier et at., filed Feb. 25, 2011; and U.S. Publication No. 2011/0245097 by Rissin et al., filed Mar. 1, 2011, each herein incorporated by reference. In some embodiments, the methods described herein may be conducted using the systems and methods described in U.S. Publication No. 2012/0196774 by Fournier et at., filed Feb. 25, 2011, herein incorporated by reference.

As will be appreciated by those in the art, a large number of analyte molecules and particles may be detected and, optionally, quantified using methods and systems of the present invention; basically, any analyte molecule that is able to be made to become immobilized with respect to a capture object can be potentially investigated using the invention. Certain more specific targets of potential interest that may comprise an analyte molecule are mentioned below. The list below is exemplary and non-limiting.

In some embodiments, the analyte molecule may be an enzyme. Non-limiting examples of enzymes include, an oxidoreductase, transferase, kinase, hydrolase, lyase, isomerase, ligase, and the like. Additional examples of enzymes include, but are not limited to, polymerases, cathepsins, calpains, amino-transferases such as, for example, AST and ALT, proteases such as, for example, caspases, nucleotide cyclases, transferases, lipases, enzymes associated with heart attacks, and the like. When a system/method of the present invention is used to detect the presence of viral or bacterial agents, appropriate target enzymes include viral or bacterial polymerases and other such enzymes, including viral or bacterial proteases, or the like.

In other embodiments, the analyte molecule may comprise an enzymatic component. For example, the analyte particle can be a cell having an enzyme or enzymatic component present on its extracellular surface. Alternatively, the analyte particle is a cell having no enzymatic component on its surface. Such a cell is typically identified using an indirect assaying method described below. Non-limiting example of enzymatic components are horseradish peroxidase, beta-galactosidase, and alkaline phosphatase.

In yet other embodiments, the analyte molecule may be a biomolecule. Non-limiting examples of biomolecules include hormones, antibodies, cytokines, proteins, nucleic acids, lipids, carbohydrates, lipids cellular membrane antigens and receptors (neural, hormonal, nutrient, and cell surface receptors) or their ligands, or combinations thereof. Non-limiting embodiments of proteins include peptides, polypeptides, protein fragments, protein complexes, fusion proteins, recombinant proteins, phosphoproteins, glycoproteins, lipoproteins, or the like. As will be appreciated by those in the art, there are a large number of possible proteinaceous analyte molecules that may be detected or evaluated for binding partners using the present invention. In addition to enzymes as discussed above, suitable protein analyte molecules include, but are not limited to, immunoglobulins, hormones, growth factors, cytokines (many of which serve as ligands for cellular receptors), cancer markers, etc. Non-limiting examples of biomolecules include PSA, TNF-alpha, troponin, and p24.

In certain embodiments, the analyte molecule may be a host-translationally modified protein (e.g., phosphorylation, methylation, glycosylation) and the capture component may be an antibody specific to a post-translational modification. Modified proteins may be captured with capture components comprising a multiplicity of specific antibodies and then the captured proteins may be further bound to a binding ligand comprising a secondary antibody with specificity to a post-translational modification. Alternatively, modified proteins may be captured with capture components comprising an antibody specific for a post-translational modification and then the captured proteins may be further bound to binding ligands comprising antibodies specific to each modified protein.

In another embodiment, the analyte molecule is a nucleic acid. A nucleic acid may be captured with a complementary nucleic acid fragment (e.g., an oligonucleotide) and then optionally subsequently labeled with a binding ligand comprising a different complementary oligonucleotide.

Suitable analyte molecules and particles include, but are not limited to small molecules (including organic compounds and inorganic compounds), environmental pollutants (including pesticides, insecticides, toxins, etc.), therapeutic molecules (including therapeutic and abused drugs, antibiotics, etc.), biomolecules (including hormones, cytokines, proteins, nucleic acids, lipids, carbohydrates, cellular membrane antigens and receptors (neural, hormonal, nutrient, and cell surface receptors) or their ligands, etc.), whole cells (including prokaryotic (such as pathogenic bacteria) and eukaryotic cells, including mammalian tumor cells), viruses (including retroviruses, herpesviruses, adenoviruses, lentiviruses, etc.), spores, etc.

The fluid sample containing or suspected of containing an analyte molecule may be derived from any suitable source. In some cases, the sample may comprise a liquid, fluent particulate solid, fluid suspension of solid particles, supercritical fluid, and/or gas. In some cases, the analyte molecule may be separated or purified from its source prior to determination; however, in certain embodiments, an untreated sample containing the analyte molecule may be tested directly. The source of the analyte molecule may be synthetic (e.g., produced in a laboratory), the environment (e.g., air, soil, etc.), a mammal, an animal, a plant, or any combination thereof. In a particular example, the source of an analyte molecule is a human bodily substance (e.g., blood, serum, plasma, urine, saliva, stool, tissue, organ, or the like). The volume of the fluid sample analyzed may potentially be any amount within a wide range of volumes, depending on a number of factors such as, for example, the number of capture objects used/available, the number of locations us/available, etc. In a few particular exemplary embodiments, the sample volume may be about 0.01 ul, about 0.1 uL, about 1 uL, about 5 uL, about 10 uL, about 100 uL, about 1 mL, about 5 mL, about 10 mL, or the like. In some cases, the volume of the fluid sample is between about 0.01 uL and about 10 mL, between about 0.01 uL and about 1 mL, between about 0.01 uL and about 100 uL, or between about 0.1 uL and about 10 uL.

In some cases, the fluid sample may be diluted prior to use in an assay. Therefore, the method may utilized a solution derived from the fluid sample. For example, in embodiments where the source of an analyte molecule is a human body fluid (e.g., blood, serum), the fluid may be diluted with an appropriate solvent (e.g., a buffer such as PBS buffer). A fluid sample may be diluted about 1-fold, about 2-fold, about 3-fold, about 4-fold, about 5-fold, about 6-fold, about 10-fold, or greater, prior to use. The sample may be added to a solution comprising the plurality of capture objects, or the plurality of capture objects may be added directly to or as a solution to the sample.

U.S. Ser. No. 62/102,818, filed Jan. 13, 2015, entitled “METHODS RELATING TO IMPROVING ACCURACY OF CAPTURE OBJECT-BASED ASSAYS” is incorporated herein by reference. U.S. patent application Ser. No. 15/543,401, filed Jul. 13, 2017, entitled “METHODS RELATING TO IMPROVING ACCURACY OF CAPTURE OBJECT-BASED ASSAYS,” and published as U.S. Patent Publication No. 2018-0003703 on Jan. 4, 2018, is incorporated herein by reference.

The following examples are included to demonstrate various features of the invention. Those of ordinary skill in the art should, in light of the present disclosure, will appreciate that many changes can be made in the specific embodiments which are disclosed while still obtaining a like or similar result without departing from the scope of the invention as defined by the appended claims. Accordingly, the following examples are intended only to illustrate certain features of the present invention, but do not necessarily exemplify the full scope of the invention

### Example 1

The following example describes a non-limiting method of determining a measure of the number of analyte molecules in a fluid sample using both targeting and non-targeting capture objects (e.g., beads). As described below, the accuracy of the assay improved with the use of both targeting and non-targeting capture objects, as compare to a substantially similar method using only targeting capture objects.

General Description of Method and Reagents:

Materials: 2.7 um (micrometer) diameter carboxyl-functionalized paramagnetic beads were purchased from Agilent Technologies. 1-Ethyl-3-(3-dimethylaminopropyl) carbodiimide hydrochloride (EDC) was purchased from Thermo Scientific. Bovine serum albumin (BSA), Dimethyl Sulfoxide (DMSO), Ethylenediaminetetraacetic acid (EDTA), 2-(N-morpholino)ethanesulfonic acid (MES), Tween20, and were purchased from Sigma-Aldrich. Phosphate buffered saline (PBS) was purchased from Amresco. Alexa Fluor 488 hydrazide and Resorufin-B-D-galactopyranoside (RGP) were purchased from Life Technologies. Cy5 Mono Hydrazide was purchased from GE Healthcare. Hilyte Fluor 750 Hydrazide was purchased from Anaspec. Antibodies and proteins standards were all purchased from commercial vendors. Detection antibodies were biotinylated using standard methods as described previously in Rissin et. al. Anal Chem, 2011, 83, 2279-2285. Streptavidin-β-Galactopyranoside (RGP) was conjugated in house using methods described previously, the average number of enzyme and streptavidin molecules per conjugate were 1.2 and 2.7, respectively, (e.g., see Rissin et. al. Anal Chem, 2011, 83, 2279-2285, herein incorporated by reference). Simoa™ discs comprised of 24 arrays of femtoliter size wells molded into cyclic olefin copolymer (COC) and bonded to a microfluidic manifold were obtained from Sony DADC. Fluorocarbon oil (Krytox®) was obtained from DuPont.

Preparation of populations of fluorescently-labeled and Ab-coated paramagnetic beads: A stock solution of paramagnetic beads (2.3×10⁹ beads/mL) was vortexed for 5 s three times, then placed on a rotary mixer for 15 minutes. 521 μL of bead solution (1.2×10⁹ beads/mL) was pipetted into a 1.7 mL polypropylene tube. The beads were separated on a magnet and washed three times with 1 mL PBS+0.1% Tween 20, and twice with 1 mL PBS. The beads were resuspended in 1 mL of PBS and transferred into a 1.7 mL polypropylene tube. 1 mg. of the dye-hydrazide was dissolved in 100 μL DMSO. A solution of 40 mg mL-1 EDC in MES buffer pH 6.2 was prepared. Sufficient PBS was first added to the tube to make the total reaction volume 1 mL. 9.4-62.1 μL of dye hydrazide solution was then added depending on the fluorescence dye choice and a level required, and 250 μL of 40 mg/mL EDC was added to the bead/dye suspension. The tube was capped, vortexed intermittently for 10 s, and placed on a rotating mixer with custom light-protective cover for 30 min. After separating the beads on a magnet, the beads were washed three times with 1 mL PBS+0.1% Tween 20, resuspended in 1 mL PBS+0.1% Tween 20, and placed on a rotating mixer for 1 h. After separating the beads from the magnet, the PBS+0.1% Tween 20 solution was removed, and the beads were resuspended in 1 mL of 100 mM sodium bicarbonate buffer and placed on a rotating mixer for 1 h. The beads were stored in 100 mM sodium bicarbonate buffer, pH 9.3 at 2-8° C. in an opaque container until conjugation with antibody. To conjugate the antibody to the dye-encoded beads, 500 μL of encoded bead stock (1.2×10⁹ beads/mL=0.600×10⁹ beads) was pipetted into a 1.7 mL polypropylene tube. The beads were separated and washed 3 times with PBS+0.1% Tween 20, followed by twice with 50 mM MES buffer pH 6.2. A solution of 0.526 mg/mL capture antibody in 50 mM MES pH 6.2 was prepared. The beads were pelleted on a magnet, the buffer was aspirated, and 0.475 mL of 0.526 mg/mL antibody solution was added to the beads. The mixture of beads and solution of antibody was vortexed, and incubated on a rotation mixer for 30 min in a custom light-protective cover. A solution containing 1 mg/mL EDC in cold 25 mM MES pH 6.0 was prepared, and 0.025 mL of this solution was added to the bead/antibody solution. This mixture was vortexed and incubated on the rotational mixer for 30 min with custom light-protective cover. After separating the beads on a magnet, the beads were washed twice with 0.5 mL PBS+0.1% Tween 20. 1 mL of 1% BSA in PBS was added to the beads and incubated for 60 min on the rotation mixer with custom light-protective cover. The beads were then washed twice with PBS+0.1% Tween 20, and stored at 2-8° C. in a buffer containing 500 mM Tris+1% BSA+0.1% Tween 20+0.15% Proclin300 antimicrobial in an opaque container until ready for use.

Capture of multiple proteins on subpopulations of magnetic beads and formation of enzyme-labeled immunocomplexes: 100 000 beads of each of the six subpopulations presenting antibodies to the six proteins were mixed, pelleted and the supernatant was aspirated. Test solutions (100 μL) were added to the mixture of the 600 000 magnetic bead beads and incubated for 35 minutes at 23° C. The beads were then separated and washed three times in 5×PBS and 0.1% Tween 20. The beads were resuspended and incubated with solutions containing mixtures of biotinylated detection antibodies (anti-IL-6 at 0.125 ug/mL; anti-TNF-α at 0.4 ug/mL; anti-GM-CSF at 0.1 ug/mL; anti-IL-10 at 0.1 ug/mL; anti-IL-1β at 0.1 ug/mL; anti-IL-1a at 0.3 ug/mL) for 5 minutes at 23° C. The beads were then separated and washed three times in 5×PBS and 0.1% Tween 20. The beads were incubated with solutions containing SβG (50 pM) for 5 min at 23° C., separated, washed seven times in 5×PBS and 0.1% Tween 20, and washed once in PBS. 600 000 beads were then resuspended in 25 μL of 100 μM RGP in PBS, and 15 μL of this bead solution was loaded into a Simoa™ disc. The bead manipulation steps and incubations were performed on a Simoa HD-1 Analyzer™.

Loading and sealing of beads in femtoliter-volume well arrays: A single molecule array disc composed of 24, 3 mm×4 mm arrays of ˜216,000 femtoliter wells and individually addressable microfluidic manifolds was loaded onto the deck of a Simoa HD-1 Analyzer™ (commercially available from Quanterix, Inc.). The disc was used in the fully automated load, seal, and imaging of the subpopulations of magnetic beads and enzyme-labeled immunocomplexes and RGP in the arrays. For each sample analyzed, 15 μL of the solution containing the mixture of bead subpopulations and RGP was pipetted by the Simoa HD-1 Analyzer™ into the inlet port of the disc. Vacuum pressure was then applied to the outlet port and drew the bead solution over the arrays of femtoliter wells. The beads were allowed to settle via gravity into the wells of the array for 90 seconds. After the beads had settled, 40 μL of fluorocarbon oil was automatically dispensed by the Simoa HD-1 Analyzer™ in the inlet port, and vacuum was simultaneously applied to the outlet port to pull the oil over the array. The oil pushed the aqueous solution and the beads that were not in the wells off of the array surface, and formed a liquid-tight seal over the wells containing beads and enzyme substrate.

Imaging of single molecules and fluorescent beads in femtoliter-volume well arrays on the Simoa HD1 Analyzer™: Once the wells were sealed using the automated load, seal, and image process, the instrument performed the imaging steps necessary for identifying which bead types were in which well, and whether enzyme activity was associated with the beads. The fluorescence-based optical system was composed of: a white light illumination source; a custom, 12-element, wide field of view (3×4 mm object) microscope lens system; a CCD camera (Allied Vision, Prosilica GT3300 8 Mp). The imaging process took 45 s in total for each array, and was composed of the following sequential steps. Initially, the array is indexed to the appropriate array and held against a reference plane to which the rest of the optical system is aligned. Next, the imaging system automatically focuses to the array by taking successive images at different focus positions using “dark field” images of the array by using the 622 nm/615 nm excitation/emission filters (exposure time=0.3 ms) and setting focus to the highest contrast image, which sets up the system for the five step image acquisition process. First, a “dark field” image of the array was acquired by using the 622 nm/615 nm excitation/emission filters (exposure time=0.3 ms). Second, an image at 574 nm/615 nm excitation/emission (exposure time=3 s) was acquired; this image is the t=0 image (F1) of the single molecule resorufin signal. Third, an image at excitation/emission of 740 nm/800 nm (exposure time=3 s) was acquired to identify beads labelled with the HF-750 dye. Fourth, an image at excitation/emission of 680 nm/720 nm (exposure time=3 s) was acquired; this image was not used in this work. Fifth, an image at excitation/emission of 574 nm/615 nm excitation/emission (exposure time=3 s) was acquired 30 s after the image F1; this image is the t=30 s image (F2) of the single molecule resorufin signal. Finally, an image at excitation/emission of 490 nm/530 nm (exposure time=2 s) was acquired to identify beads labelled with the AF-488 dye. Images were saved as a single IPL file.

Analysis of Images: A custom image analysis software program was used to determine the enzyme activity associated with each bead within each subpopulation from the captured images. An algorithm first identified and removed occlusions (such as bubbles and dust) from the images. A masking method was then applied to the dark field image to define the locations and boundaries of the wells. The resulting well mask was then applied to each of the fluorescence images to determine the presence of beads and enzymes within the wells. For the bead fluorescence images, histograms of fluorescence intensity were generated for the well population. Peaks in the histograms were identified automatically and used to determine the populations of empty wells (low fluorescence), and populations of single beads at a particular fluorescence level for each fluorescence wavelength. The well mask was also applied to the difference between the second and first frame at the resorufin wavelengths, i.e., F2−F1. Wells that had been classified as containing a single bead from a particular bead subpopulation were classified as: a) associated with enzyme activity (“on” or active), if the fluorescence from resorufin within that well increased beyond a known threshold, or; b) not associated with enzyme activity (“off” or inactive), if the fluorescence from resorufin within that well did not increase beyond a known threshold. For each “on” bead the intensity increase was determined. For each bead subpopulation, the fraction of “on” beads (fon) was determined. In the digital range (fₒₙ<0.7), fₒₙ was converted to average number of enzymes per bead (AEB) using the Poisson distribution equation as described previously. In the analog range (fₒₙ>0.7), AEB was determined from the average increase in fluorescence of all the beads in an array. During classification of beaded wells and determination of enzyme activity, the fluorescence and location of wells were corrected for the following: optical blurring and scattering, background non-uniformity, intra-well bead settling locations, wavelength-dependent refraction differences in the lens assembly, and bleed of fluorescence of dyes outside their dominant wavelengths.

Results and Discussion: In the non-limiting method described in this example, it was determined that the amount of beads initially added to the sample for target analyte capture can be both advantageous and disadvantageous with respect to changes in sensitivity. For example, when using fewer beads, the ratio of target analyte to beads increases, the signal (average enzyme (e.g., target analyte) per bead or AEB) increases, and therefore, the assay sensitivity increases. However, using fewer beads also reduces the number of beads that may be loaded into the single molecule array and, if that number drops to a level where Poisson noise becomes significant, then the quantitation of beads becomes noisy and sensitivity can be decreased. The use of both targeting and non-targeting capture object counters this by allowing the minimum number of beads to be used to increase AEB while keeping the bead loading number as high and consistent as possible.

In this example, the number of target beads for a particular analyte loaded into the arrays was held at a fixed number of input beads (e.g., 100,000), and the number of non-targeting beads was varied (e.g., 0 to 500,000). The assay provided more consistent results in the presence of the non-targeting beads as compared to in the absence of the non-targeting beads. The targeting beads were distinguished from the non-targeting beads by use of fluorescence dyes.

In a first non-limiting example wherein the target analyte was IL-1alpha, 100,000 IL-1alpha targeting beads per assay sample were utilized with no other beads added (e.g., with no non-targeting beads), the average number of targeting beads detected was 2,622 per sample with a coefficient of variation (CV) of 37%. Furthermore, 6 samples did not have sufficient beads loaded to allow determination of bead number. Using 100,000 targeting IL-1alpha beads and 500,000 non-targeting beads (e.g., 100,000 each of beads specific to IL-6, TNF-alpha, GM-CSF, IL-10, IL-1beta) that could be distinguished by fluorescent the average number of targeting beads detected was 3,127 with a CV of 10%. Alexa Fluor 488 hydrazide (AF-488), cyanine 5 hydrazide (cy5), and Hilyte Fluor 750 hydrazide (HF-750) dyes were used to encode bead types for multiplexed digital ELISA. By precisely controlling the ratio of encoding dye molecules to beads, discrete encoding levels for each dye were prepared, yielding subpopulations of beads that can be distinguished on the Simoa HD1 Analyzer™. An algorithm first identified and removed occlusions (such as bubbles and dust) from the images. A masking method was then applied to the dark field image to define the locations and boundaries of the wells. The resulting well mask was then applied to each of the fluorescence images to determine the presence of beads and enzymes within the wells. All samples had sufficient beads loaded in this case. The 3-fold improvement in the precision of the targeting bead loaded greatly improves the reliability of quantifying AEB at these low bead numbers (100,000 per sample). See Table 1 for tabulated data.

TABLE 1
Comparison of number of targeting beads
loaded for 24 samples each of 100,000 beads
coated with anti-IL-1alpha alone and with
500,000 of other non-targeting
bead types present.

| | 100,000 | 100,000 IL-1alpha |
|---|---|---|
| | IL-1alpha | beads + 500,000 of |
| Sample | beads alone | non-targeting beads |
| 1 | | 3297 |
| 2 | 2580 | 3435 |
| 3 | 3971 | 3361 |
| 4 | 2542 | 3195 |
| 5 | 2792 | 3395 |
| 6 | 3153 | 3210 |
| 7 | 2275 | 2921 |
| 8 | 1210 | 2876 |
| 9 | | 2916 |
| 10 | 3336 | 2629 |
| 11 | 2784 | 3278 |
| 12 | | 2951 |
| 13 | 1697 | 3631 |
| 14 | 711 | 3362 |
| 15 | 2836 | 3200 |
| 16 | 4293 | 2706 |
| 17 | 3064 | 3507 |
| 18 | 2806 | 2414 |
| 19 | 3711 | 3626 |
| 20 | | 3246 |
| 21 | 2348 | 2978 |
| 22 | | 2949 |
| 23 | 1089 | 2869 |
| 24 | | 3100 |
| Average | 2622 | 3127 |
| number | | |
| of beads | | |
| CV | 37% | 10% |

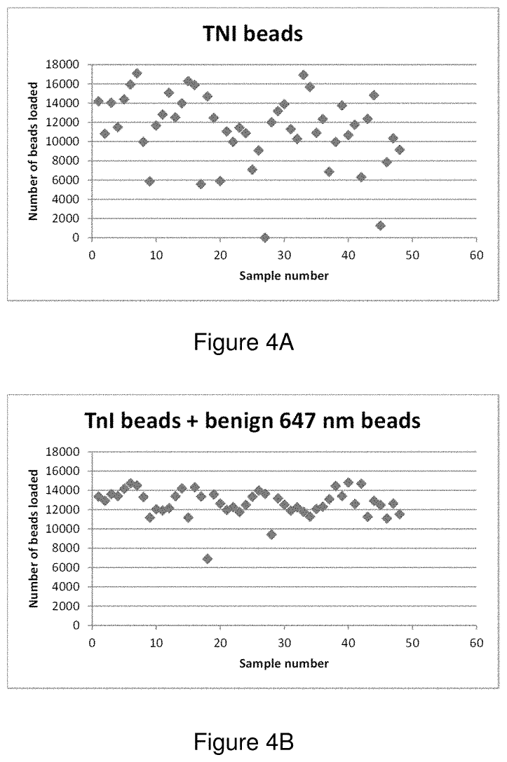

As a second non-limiting example, wherein the target analyte was TnI, target beads coated in a specific antibody to troponin I (TnI) were utilized. Using 300,000 target beads per sample, the average number of targeting beads detected was 11,284 per sample with a coefficient of variation (CV) of 33%. Using 300,000 target beads plus 300,000 of non-targeting beads that did not present any antibodies but could be distinguished from the target beads by a dye that fluoresces at 647 nm, the average number of targeting beads detected was 12,664 with a CV of 11% (see FIG. 4 and Table 2). The plots of average number of targeting beads detected in the two cases shows the dramatic improvement in the variability of bead loading by inclusion of the non-targeting beads.

In FIG. 4: Comparison of the average number of targeting beads detected over 48 samples using: a) 300,000 targeting TnI beads (left); and b) 300,000 targeting TnI beads+300,000 647 nm-labeled non-targeting beads.

TABLE 2
Comparison of signals (AEB) and imprecision (CV)
as a function of TnI concentration for targeting TnI
beads alone and combined with non-targeting beads.

| | | | 300,000 targeting TnI | |
|---|---|---|---|---|
| | 300,000 targeting | | beads + 300,000 non- | |
| [TnI] | TnI beads alone | | targeting 647 nm beads | |
| (pg/mL) | AEB | CV | AEB | CV |
| 0 | 0.00903314 | 25% | 0.009094454 | 8% |
| 0.1 | 0.01774402 | 6% | 0.017927884 | 13% |
| 0.3 | 0.03279242 | 17% | 0.034686352 | 10% |
| 1 | 0.10172504 | 20% | 0.10539009 | 11% |
| 3 | 0.22942951 | 39% | 0.275594076 | 9% |
| 10 | 0.89510673 | 15% | 0.947662845 | 13% |
| 30 | 2.88942545 | 5% | 2.66543617 | 7% |
| 100 | 8.37164458 | 8% | 8.409316094 | 8% |

### Example 2

In this example, non-target capture beads were used to improve the reproducibility of bead fill and AEB in an assay for troponin I (TnI). Single molecule assays were running on three Simoa HD-1 Analyzers™ (VU 15, 16, and 18) using 300,000 target capture TnI-specific beads with and without 300,000 non-target dye-labeled beads. As can be seen from Table 3 and the associated plots of bead fill for each instrument, the precision of bead fill (quantified by CV of bead fill) was improved on all three instruments by using non-target capture beads. As a result of more reliable bead fill, the instrument-to-instrument variability of AEB values of TnI at various concentrations in a calibration curve was reduced.

TABLE 3

| | Ex. 1 | | Ex. 2 | | Ex. 3 | |
|---|---|---|---|---|---|---|
| | w/o non- | w/non- | w/o non- | w/non- | w/o non- | w/non- |
| | target | target | target | target | target | target |
| | beads | beads | beads | beads | beads | beads |
| Beads fill % | 7.7 | 9.0 | 8.9 | 9.4 | 10.0 | 9.3 |
| average | | | | | | |
| Beads fill | 32% | 11% | 19% | 10% | 12% | 10% |
| CV | | | | | | |

### Example 3

In this example, non-targeting capture objects (e.g., non-targeting beads) were used to provide signal normalization. That is, an array specific measurement of I_{single} using the non-targeting beads was obtained to account for assay variations, including but not limited to temperature, labeling reagent concentrations, enzyme activity, and well depth. As noted herein, I_{single} calculation for every array may be useful and more accurate as compared to using average I_{single} values from previously analyzed arrays, e.g., stored calibration curves. As noted, any changes in the environment that affect the velocity of substrate turnover may yield inaccurate results for analog samples (fₒₙ of >0.7)) that are processed at a different time than the previously analyzed arrays used to determine the average I_{single} value, e.g., from a calibration curve. The reason for this potential inaccuracy may be caused because those analog samples are converted to AEB from the I_{single} value calculated from the stored calibration curve that may have been generated under different environmental conditions.

Method Overview: A two-bead assay format was utilized, wherein the first type of bead was an assay-specific bead used to target the target analyte, and the second type of bead was a non-targeting bead used to determine I_{single} for every sample/array. Each of the two types of beads were encoded with a unique dye barcode so image analysis was used to determine which type of bead was in each location. The non-target beads were used to bind a statistically significant number of single molecules to enable accurate and precise calculation of I_{single}, but not too many single molecules such that the fₒₙ value would be so high that the I_{single} would be inaccurately determined. In order to assure that an optimal number of single molecules were bound to the non-target beads, the non-target beads were associated with a low concentration of biotin molecules after the dye encoding process. Experiments were conducted to determine the amount of biotin that the non-target beads should be functionalized with to yield f₍ₒₙ₎ between 0.05 and 0.20 (see below). The protocols outlined below yielded 488 nm-encoded non-target beads. These non-target beads can be combined with assay beads encoded with other encoding dyes or combination of dyes, for example, that are decoded with the 647 nm, 700 nm, and 750 nm optical channels. It should be understood that the non-target beads could also be encoded with any dye and at any dye level, but generally, the non-target beads are distinguishable from the target beads.

Preparation of 488 encoded beads. 488-glycine amide dye was dissolved in DMSO to 10 mg/mL and diluted in phosphate buffered saline (PBS) to 1 mg/mL. 1.2×10⁹ 2.7-micron diameter paramagnetic beads/mL was pipetting into the solution. The beads were washed three times in PBS and 0.1% Tween 20. The beads were then washed three times in PBS. The beads were resuspended in 971.76 uL PBS per mL of the batch was added. Added 3.24 uL/mL of the batch size of 1 mg/mL dye stock solution, which was then incubated on a mixer for 5 min. 25 ul/mL of the batch size was added to 40 mg/mL EDC. This was vortexed ten times and placed on mixer for 30 min. The beads were washed three times in PBS and 0.1% Tween 20 and then incubated for 1 hour. 1 ml/mL Sodium Bicarbonate Buffer pH 9.3 was added to the solution.

Preparation of biotin-functionalized 488-nm encoded beads. Hydrazide biotin was dissolved in DMSO to 10 mg/mL. 1.2×10⁹ 2.7-micron diameter paramagnetic beads/mL were added. The beads were washed three times in PBS and 0.1% Tween 20. The beads were then washed three times PBS. The beads were resuspended in 962.2 uL PBS per mL of the batch size. 12.8 uL of 1:10,000 dilution of 10 mg/mL biotin/DMSO solution was added. This was incubated on mixer for 30 min. Added 25 uL/mL batch size of 40 mg/mL EDC dissolved in 50 mM MES pH 6.2, which was then vortexed ten times and placed on mixer for 30 min. The beads were then washed three times PBS and 0.1% Tween 20. 1 mL 1% bovine serum albumin (BSA) per mL of the batch size was added, followed by incubation for 1 hour. The beads were washed three times in PBS and 0.1% Tween 20. 1 mL of bead storage buffer was added per 1M1 of the batch size for storage.

Protocol for preparing the two-bead solution and running the assay: 4×10⁶ beads/mL of 750-nm encoded PSA beads and 4×10⁶/mL of 488-nm encoded non-target capture normalization beads were prepared in bead diluent. The resulting number of beads per cuvette was 400,000 PSA beads and 400,000 non-target beads. The assay was performed using the standard PSA assay reagents and conditions, with the only difference being the bead reagent containing the non-target capture normalization beads. The beads were incubated with 100-μL samples containing known concentrations of PSA (shown in Table 3) for 15 min. The beads were washed 3 times in wash buffer and then incubated for 5 min with 100 μL of anti-PSA detection antibody (0.328 μg/mL). The beads were washed 3 times in wash buffer and then incubated for 5 min with 100 μL of streptavidin-beta-galactosidase (50 pM). The beads were washed 6 times with wash buffer, resuspended in RGP substrate, load, sealed and imaged on the Simoa HD-1 Analyzer™. Table 4 shows results from the same experiment analyzed, either with or without using the non-targeting capture beads to determine I_{single}.

In this example, AEB (digital) was determined for arrays with fₒₙ<0.76, and AEB (analog) was determined for arrays with fₒₙ>0.76, for 7 concentrations of PSA in quadruplicate. Table 5 summarizes the average AEB (both digital and analog) and standard deviation and coefficient of variation

TABLE 4

| | | | Isingle of target | Isingle of within | | AEB (analog) | AEB (analog) |
|---|---|---|---|---|---|---|---|
| | | | capture beads | array non-target | | using target | using within- |
| [PSA] | | | in all arrays with | capture beads | AEB | beads batch | array non-target |
| (pg/mL) | I(bead) | f(on) | fon < 0.2 in batch | with fon < 0.2 | (digital) | Isingle | beads Isingle |
| 0.1 | 123.6 | 0.037 | 91.3 | 95.9 | 0.038 | | |
| 0.1 | 103.3 | 0.037 | 91.3 | 89.2 | 0.038 | | |
| 0.1 | 104.3 | 0.039 | 91.3 | 84.5 | 0.040 | | |
| 0.1 | 91.9 | 0.035 | 91.3 | 85.7 | 0.036 | | |
| 0.3 | 93.6 | 0.084 | 91.3 | 78.8 | 0.088 | | |
| 0.3 | 91.0 | 0.090 | 91.3 | 85.3 | 0.094 | | |
| 0.3 | 86.3 | 0.089 | 91.3 | 78.3 | 0.094 | | |
| 0.3 | 89.6 | 0.085 | 91.3 | 83.9 | 0.089 | | |
| 1 | 95.1 | 0.224 | 91.3 | 79.2 | 0.254 | | |
| 1 | 99.1 | 0.223 | 91.3 | 83.2 | 0.252 | | |
| 1 | 96.6 | 0.215 | 91.3 | 83.0 | 0.241 | | |
| 1 | 96.9 | 0.225 | 91.3 | 82.2 | 0.255 | | |
| 3 | 119.7 | 0.509 | 91.3 | 83.4 | 0.711 | | |
| 3 | 124.0 | 0.528 | 91.3 | 81.7 | 0.751 | | |
| 3 | 125.1 | 0.520 | 91.3 | 83.0 | 0.735 | | |
| 3 | 123.5 | 0.511 | 91.3 | 86.5 | 0.715 | | |
| 10 | 233.0 | 0.872 | 91.3 | 84.8 | | 2.227 | 2.396 |
| 10 | 233.9 | 0.884 | 91.3 | 83.7 | | 2.266 | 2.472 |
| 10 | 234.2 | 0.896 | 91.3 | 90.5 | | 2.299 | 2.320 |
| 10 | 235.9 | 0.897 | 91.3 | 84.7 | | 2.318 | 2.499 |
| 30 | 597.0 | 0.975 | 91.3 | 87.7 | | 6.379 | 6.638 |
| 30 | 650.8 | 0.991 | 91.3 | 92.4 | | 7.065 | 6.978 |
| 30 | 601.0 | 0.963 | 91.3 | 87.1 | | 6.339 | 6.641 |
| 30 | 633.1 | 0.989 | 91.3 | 89.6 | | 6.863 | 6.994 |
| 100 | 1425.9 | 0.999 | 91.3 | 95.8 | | 15.614 | 14.869 |
| 100 | 1359.5 | 0.998 | 91.3 | 94.8 | | 14.864 | 14.314 |
| 100 | 1368.7 | 0.999 | 91.3 | 94.6 | | 14.987 | 14.463 |
| 100 | 1404.2 | 0.991 | 91.3 | 96.0 | | 15.243 | 14.488 |

TABLE 5

| | Based on I_{single} from batch | | | Based on I_{single} from within- | | |
|---|---|---|---|---|---|---|
| | I_{single} of target beads in arrays | | | array non-target beads with | | |
| | with f(on) < 0.2 | | | f(on) < 0.2 | | |
| [PSA] | | Standard | CV | | Standard | CV |
| (pg/mL) | AEB | Deviation | (%) | AEB | Deviation | (%) |
| 0.1 | 0.038 | 0.002 | 4.8% | 0.038 | 0.002 | 4.8% |
| 0.3 | 0.091 | 0.003 | 3.4% | 0.091 | 0.003 | 3.4% |
| 1 | 0.251 | 0.006 | 2.5% | 0.251 | 0.006 | 2.5% |
| 3 | 0.728 | 0.018 | 2.5% | 0.728 | 0.018 | 2.5% |
| 10 | 2.277 | 0.040 | 1.8% | 2.422 | 0.081 | 3.3% |
| 30 | 6.661 | 0.359 | 5.4% | 6.813 | 0.200 | 2.9% |
| 100 | 15.177 | 0.331 | 2.2% | 14.534 | 0.236 | 1.6% |

### Example 4

The following example utilizes substantially similar procedures as described in Example 3, except that instead of using biotin-presenting non-targeting capture objects, non-targeting capture objects were prepared that were coated with an antibody to beta-galactosidase (anti-beta-gal). The loading of anti-beta-gal on these beads was optimized to ensure that the fₒₙ of these beads in the presence of streptavidin-beta-galactosidase was <0.2, so that single molecules predominated on the non-target capture beads and precise I_{single} values could be determined within each array. These experiments were performed in a laboratory held at a temperature of 25 degrees C., and four concentrations of PSA (0, 0.1, 30, and 100 pg/mL) were tested. Table 6 shows the I_{single} values determined from the target capture beads. Table 7 shows the AEB values in these experiments, where the analog values of AEB (>1.2) were calculated either using the batch I_{single} from target capture beads or the I_{single} from within array non-target capture beads.

TABLE 6

| | Isingle from | | | | | | | | | |
|---|---|---|---|---|---|---|---|---|---|---|
| | target capture | | Isingle of within array non-target capture beads with | | | | | | | |
| | beads with | | f(on) < 0.2 | | | | | | | |
| | f(on) < 0.2 | | 0 pg/mL | | 0.1 pg/mL | | 30 pg/mL | | 100 pg/mL | |
| | Mean | SD | Mean | SD | Mean | SD | Mean | SD | Mean | SD |
| 25° C. | 276.7 | 16.6 | 267.2 | 15.9 | 271.4 | 17.2 | 275.9 | 9.5 | 274.5 | 1.8 |

TABLE 7

| | 25° C. | | | |
|---|---|---|---|---|
| | AEB(analog) | | AEB(analog) | |
| | calculated using | | calculated using | |
| | non-target capture | | target beads batch | |
| [PSA] | beads Isingle | | Isingle | |
| (pg/mL) | Mean | SD | Mean | SD |
| 0 | 0.026242 | 0.00921 | 0.026242 | 0.00921 |
| 0.1 | 0.049648 | 0.011267 | 0.049648 | 0.011267 |
| 30 | 2.489554 | 0.568742 | 2.479028 | 0.566931 |
| 100 | 8.43896 | 1.595863 | 8.394636 | 1.691394 |

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively.

1. A method for measuring a concentration of an analyte in a fluid sample, comprising:
exposing analyte capture objects and non-targeting capture objects, present in a ratio of a number of analyte capture objects to a sum of the analyte capture objects and non-targeting capture objects of between 1:1.2 and 1:100, to the fluid sample or a solution derived from the fluid sample;
wherein the analyte capture objects have specific binding affinity for the analyte;
wherein the non-targeting capture objects do not have specific binding affinity for any analyte contained in or suspected to be contained in the fluid sample;
wherein only some of the analyte capture objects associate with the analyte;
partitioning at least some of the capture objects exposed to the fluid sample or the solution derived from the fluid sample into separate locations;
interrogating at least some of the locations to determine which locations contain an analyte capture object and in which of such locations the analyte capture object is bound to analyte; and
measuring a concentration of the analyte in the fluid sample at least in part based on a ratio of a number of locations interrogated containing an analyte capture object bound to analyte to a total number of locations interrogated containing an analyte capture object.

2. The method of claim 1, wherein the analyte capture objects and the non-targeting capture objects are beads.

3. The method of claim 2, wherein the beads have an average diameter of between 0.1 micrometer and 100 micrometers.

4. The method of claim 2, wherein the beads have an average diameter of between 1 micrometer and 10 micrometers.

5. The method of claim 1, wherein the exposing the capture objects to the fluid sample or a solution derived from the fluid sample comprises suspending the capture objects in the fluid sample or the solution derived from the fluid sample.

6. The method of claim 1, wherein the plurality of locations comprise a plurality of reaction vessels.

7. The method of claim 6, wherein the average volume of the plurality of reaction vessels is between 10 attoliters and 100 picoliters.

8. The method of claim 6, wherein the average volume of the plurality of reaction vessels is between 1 femtoliter and 1 picoliter.

9. The method of claim 1, wherein the interrogating at least some of the locations uses optical techniques.

10. The method of claim 1, wherein the analyte comprises a protein.

11. The method of claim 1, wherein the analyte comprises a nucleic acid.

12. The method of claim 1, wherein the analyte capture objects and the non-targeting capture objects are present in a ratio of a number of analyte capture objects to a sum of the analyte capture objects and non-targeting capture objects of between 1:2 and 1:100.

13. The method of claim 1, wherein the non-targeting capture objects are not considered in the measuring step.

14. The method of claim 1, wherein the fluid sample is sourced from the environment, an animal, a plant, or any combination thereof.

15. The method of claim 1, wherein the fluid sample is sourced from a human bodily substance.

16. The method of claim 1, wherein each of the non-targeting beads is free of antibodies.

17. The method of claim 1, wherein each of the non-targeting beads is free of proteins.

18. The method of claim 1, wherein each of the non-targeting beads is free of capture object surface-bound molecules.

19. The method of claim 1, wherein each of the non-targeting beads is functionalized with antibodies lacking specific binding affinity for the analyte and any molecule known to be or suspected of being present in the fluid sample or the solution derived from the fluid sample.

20. The method of claim 1, wherein:
the analyte capture objects and the non-targeting capture objects are beads having an average diameter of between 1 micrometer and 10 micrometers;
the plurality of locations comprise a plurality of reaction vessels having an average volume of between 1 femtoliter and 1 picoliter;
the exposing the capture objects to the fluid sample or a solution derived from the fluid sample comprises suspending the capture objects in the fluid sample or the solution derived from the fluid sample;
the analyte comprises a protein;
the non-targeting capture objects are not considered in the measuring step; and
the fluid sample is sourced from a human bodily substance.
